SEQUENCE LISTING (94 sequences)

SEQ ID NO: 1 - protein, 12 aa, Artificial Sequence
  CDRH1
GFSLSTYGVGVG

SEQ ID NO: 2 - protein, 16 aa, Artificial Sequence
  CDRH2
NIWWDDDKRYNPSLEN

SEQ ID NO: 3 - protein, 13 aa, Artificial Sequence
  CDRH3
TPAYYGSHPPFDY

SEQ ID NO: 4 - protein, 11 aa, Artificial Sequence
  CDRL1
RTSEDIYTNLA

SEQ ID NO: 5 - protein, 7 aa, Artificial Sequence
  CDRL2
VAKTLQD

SEQ ID NO: 6 - protein, 9 aa, Artificial Sequence
  CDRL3
LQGFKFPWT

SEQ ID NO: 7 - protein, 7 aa, Artificial Sequence
  CDRL2 VARIANT
VAKTLQE

SEQ ID NO: 8 - protein, 107 aa, Artificial Sequence
  1638 gL7 V-region
DIQMTQSPSSLSASVGDRVTITCRTSEDIYTNLAWYQQKPGKVPKLLIYVAKTLQEGVPSRFSGSGSGTHYTLTISSLQPEDVATYYCLQGFKFPWTFGGGTKVEIK

SEQ ID NO: 9 - protein, 123 aa, Artificial Sequence
  1638 gH33 V-region
EVQLVESGGGLVQPGGSLRLSCAASGFSLSTYGVGVGWVRQAPGKGLEWLANIWWDDDKRYNPSLENRFTISRDNAKNSAYLQMNSLRAEDTAVYYCARTPAYYGSHPPFDYWGQGTMVTVSS

SEQ ID NO: 10 - protein, 214 aa, Artificial Sequence
  1638 gL7 Light chain
DIQMTQSPSSLSASVGDRVTITCRTSEDIYTNLAWYQQKPGKVPKLLIYVAKTLQEGVPSRFSGSGSGTHYTLTISSLQPEDVATYYCLQGFKFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 642 bp, Artificial Sequence
  DNA encoding gL7 Light chain
gatatccagatgacccagagtccaagcagtctctccgccagcgtaggcgatcgtgtgactattacctgtcgcactagcgaggacatctacaccaacctggcgtggtatcagcagaaaccaggcaaagtgccgaaactgctgatctacgtcgcgaaaaccctccaggaaggtgtaccgtctcgcttttccggctctggtagcggtactcactacaccctgaccatctcttccctccagccggaagatgttgctacctactattgcctccagggcttcaaattcccgtggactttcggtggcggcacgaaagtggaaatcaaacggaccgtggccgctccctccgtgttcatcttcccaccctccgacgagcagctgaagtccggcaccgcctccgtcgtgtgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgc

SEQ ID NO: 12 - protein, 490 aa, Artificial Sequence
  1638 gH33 Fab-dsscFv (1735h5.hFab-scFv.768 ) heavy chain amino acid sequence
EVQLVESGGGLVQPGGSLRLSCAASGFSLSTYGVGVGWVRQAPGKGLEWLANIWWDDDKRYNPSLENRFTISRDNAKNSAYLQMNSLRAEDTAVYYCARTPAYYGSHPPFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCSGGGGTGGGGSEVQLLESGGGLVQPGGSLRLSCAVSGIDLSNYAINWVRQAPGKCLEWIGIIWASGTTFYATWAKGRFTISRDNSKNTVYLQMNSLRAEDTAVYYCARTVPGYSTAPYFDLWGQGTLVTVSSGGGGSGGGGSGGGGSGGGGSDIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGCGTKVEIKRT

SEQ ID NO: 13 - DNA, 1470 bp, Artificial Sequence
  DNA encoding 1638 gH33 Fab-dsscfv (1735h5.hFab-scFv.768 ) Heavy chain
gaggttcagctggtcgagtctggaggcgggcttgtccagcctggagggagcctgcgtctctcttgtgcagcgtccggcttctctctgtctacctacggcgttggtgttggttgggtacgtcaggctccaggtaaaggtctggaatggctcgcaaacatctggtgggacgacgataaacgctacaacccgtccctggagaaccgcttcaccattagccgtgataacgcgaaaaactccgcgtatctccagatgaactccctgcgtgccgaagacacggctgtgtactattgcgcgcgcactccggcgtactatggctctcacccaccgtttgattactggggtcagggtacaatggttaccgtctcgtctgcctccaccaagggcccatcggtcttccccctggcaccctcctccaagagcacctctgggggcacagcggccctgggctgcctggtcaaggactacttccccgaaccagtgacggtgtcgtggaactcaggtgccctgaccagcggcgttcacaccttcccggctgtcctacagtcttcaggactctactccctgagcagcgtggtgaccgtgccctccagcagcttgggcacccagacctacatctgcaacgtgaatcacaagcccagcaacaccaaggtcgataagaaagttgagcccaaatcttgtagtggaggtgggggcaccggtggaggtggcagcgaggttcaactgcttgagtctggaggaggcctagtccagcctggagggagcctgcgtctctcttgtgcagtaagcggcatcgacctgagcaattacgccatcaactgggtgagacaagctccggggaagtgtttagaatggatcggtataatatgggccagtgggacgaccttttatgctacatgggcgaaaggaaggtttacaattagccgggacaatagcaaaaacaccgtgtatctccaaatgaactccttgcgagcagaggacacggcggtgtactattgtgctcgcactgtcccaggttatagcactgcaccctacttcgatctgtggggacaagggaccctggtgactgtttcaagtggcggagggggtagtggagggggtggctctgggggtggcggaagcggtggcgggggttctgacatacaaatgactcagtctccttcatcggtatccgcgtccgttggcgatagggtgactattacatgtcaaagctctcctagcgtctggagcaattttctatcctggtatcaacagaaaccggggaaggctccaaaacttctgatttatgaagcctcgaaactcaccagtggagttccgtcaagattcagtggctctggatcagggacagacttcacgttgacaatcagttcgctgcaaccagaggactttgcgacctactattgtggtggaggttacagtagcataagtgatacgacatttgggtgcggtactaaggtggaaatcaaacgtacc

SEQ ID NO: 14 - protein, 226 aa, Artificial Sequence
  1638 gH33 Heavy chain
EVQLVESGGGLVQPGGSLRLSCAASGFSLSTYGVGVGWVRQAPGKGLEWLANIWWDDDKRYNPSLENRFTISRDNAKNSAYLQMNSLRAEDTAVYYCARTPAYYGSHPPFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 15 - protein, 121 aa, Artificial Sequence
  Albumin binding dsscFv heavy chain
EVQLLESGGGLVQPGGSLRLSCAVSGIDLSNYAINWVRQAPGKCLEWIGIIWASGTTFYATWAKGRFTISRDNSKNTVYLQMNSLRAEDTAVYYCARTVPGYSTAPYFDLWGQGTLVTVSS

SEQ ID NO: 16 - protein, 112 aa, Artificial Sequence
  Albumin binding dsscFv light chain
DIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGCGTKVEIKRT

SEQ ID NO: 17 - protein, 274 aa, Artificial Sequence
  Cyno FcRn alpha chain extracellular sequence
AESHLSLLYHLTAVSSPAPGTPAFWVSGWLGPQQYLSYDSLRGQAEPCGAWVWENQVSWYWEKETTDLRIKEKLFLEAFKALGGKGPYTLQGLLGCELSPDNTSVPTAKFALNGEEFMNFDLKQGTWGGDWPEALAISQRWQQQDKAANKELTFLLFSCPHRLREHLERGRGNLEWKEPPSMRLKARPGNPGFSVLTCSAFSFYPPELQLRFLRNGMAAGTGQGDFGPNSDGSFHASSSLTVKSGDEHHYCCIVQHAGLAQPLRVELETPAKSS

SEQ ID NO: 18 - protein, 99 aa, Artificial Sequence
  Cyno B2-microglobulin
IQRTPKIQVYSRHPPENGKPNFLNCYVSGFHPSDIEVDLLKNGEKMGKVEHSDLSFSKDWSFYLLYYTEFTPNEKDEYACRVNHVTLSGPRTVKWDRDM

SEQ ID NO: 19 - protein, 107 aa, Artificial Sequence
  Human IGKV1-27 JK4 acceptor framework
DIQMTQSPSSLSASVGDRVTITCRASQGISNYLAWYQQKPGKVPKLLIYAASTLQSGVPSRFSGSGSGTDFTLTISSLQPEDVATYYCQKYNSAPLTFGGGTKVEIK

SEQ ID NO: 20 - protein, 114 aa, Artificial Sequence
  Human IGHV3-7 JH3 acceptor framework
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARDAFDVWGQGTMVTVSS

SEQ ID NO: 21 - protein, 274 aa, Artificial Sequence
  Human FcRn alpha chain extracellular sequence
AESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSS

SEQ ID NO: 22 - protein, 99 aa, Artificial Sequence
  Rat B2M
IQKTPQIQVYSRHPPENGKPNFLNCYVSQFHPPQIEIELLKNGKKIPNIEMSDLSFSKDWSFYILAHTEFTPTETDVYACRVKHVTLKEPKTVTWDRDM

SEQ ID NO: 23 - protein, 119 aa, Artificial Sequence
  Human B2M including signal sequence
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 24 - protein, 99 aa, Artificial Sequence [sequence omitted]

SEQ ID NO: 25 - protein, 107 aa, Artificial Sequence
  1638gL2 V
DIQMTQSPSSLSASVGDRVTITCRTSEDIYTNLAWYQQKPGKVPKLLIYVAKTLQDGVPSRFSGSGSGTHYTLTISSLQPEDVATYYCLQGFKFPWTFGGGTKVEIK

SEQ ID NO: 26 - protein, 214 aa, Artificial Sequence
  1638 gL2 light chain (V + constant)
DIQMTQSPSSLSASVGDRVTITCRTSEDIYTNLAWYQQKPGKVPKLLIYVAKTLQDGVPSRFSGSGSGTHYTLTISSLQPEDVATYYCLQGFKFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 123 aa, Artificial Sequence
  1638gH2 V-region
EVQLVESGGGLVQPGGSLRLSCAFSGFSLSTYGVGVGWVRQAPGKGLEWLANIWWDDDKRYNPSLENRFTISKDTAKNSAYLQMNSLRAEDTAVYYCVRTPAYYGSHPPFDYWGQGTMVTVSS

SEQ ID NO: 28 - protein, 226 aa, Artificial Sequence
  1638 gH2 Fab heavy chain (V + human gamma-1 CH1 no hinge)
EVQLVESGGGLVQPGGSLRLSCAFSGFSLSTYGVGVGWVRQAPGKGLEWLANIWWDDDKRYNPSLENRFTISKDTAKNSAYLQMNSLRAEDTAVYYCVRTPAYYGSHPPFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 29 - protein, 5 aa, Artificial Sequence
  linker
GGGGS

SEQ ID NO: 30 - protein, 20 aa, Artificial Sequence
  linker
GGGGSGGGGSGGGGSGGGGS

SEQ ID NO: 31 - protein, 21 aa, Artificial Sequence
  linker
SGGGGSGGGGSGGGGSGGGGS

SEQ ID NO: 32 - protein, 16 aa, Artificial Sequence
  linker
SGGGGSGGGGTGGGGS

SEQ ID NO: 33 - protein, 11 aa, Artificial Sequence
  linker
SGGGGTGGGGS

SEQ ID NO: 34 - protein, 8 aa, Artificial Sequence
  Hinge linker
DKTHTCAA

SEQ ID NO: 35 - protein, 11 aa, Artificial Sequence
  Hinge linker
DKTHTCPPCPA

SEQ ID NO: 36 - protein, 18 aa, Artificial Sequence
  Hinge linker
DKTHTCPPCPATCPPCPA

SEQ ID NO: 37 - protein, 25 aa, Artificial Sequence
  Hinge linker
DKTHTCPPCPATCPPCPATCPPCPA

SEQ ID NO: 38 - protein, 30 aa, Artificial Sequence
  Hinge linker
DKTHTCPPCPAGKPTLYNSLVMSDTAGTCY

SEQ ID NO: 39 - protein, 31 aa, Artificial Sequence
  Hinge linker
DKTHTCPPCPAGKPTHVNVSVVMAEVDGTCY

SEQ ID NO: 40 - protein, 15 aa, Artificial Sequence
  Hinge linker
DKTHTCCVECPPCPA

SEQ ID NO: 41 - protein, 26 aa, Artificial Sequence
  Hinge linker
DKTHTCPRCPEPKSCDTPPPCPRCPA

SEQ ID NO: 42 - protein, 11 aa, Artificial Sequence
  Hinge linker
DKTHTCPSCPA

SEQ ID NO: 43 - protein, 7 aa, Artificial Sequence
  Flexible linker
SGGGGSE

SEQ ID NO: 44 - protein, 6 aa, Artificial Sequence
  Flexible linker
DKTHTS

SEQ ID NO: 45 - protein, 6 aa, Artificial Sequence
  Flexible linker
SGGGGS

SEQ ID NO: 46 - protein, 11 aa, Artificial Sequence
  Flexible linker
SGGGGSGGGGS

SEQ ID NO: 47 - protein, 16 aa, Artificial Sequence
  Flexible linker
SGGGGSGGGGSGGGGS

SEQ ID NO: 48 - protein, 21 aa, Artificial Sequence
  Flexible linker
SGGGGSGGGGSGGGGSGGGGS

SEQ ID NO: 49 - protein, 26 aa, Artificial Sequence
  Flexible linker
SGGGGSGGGGSGGGGSGGGGSGGGGS

SEQ ID NO: 50 - protein, 11 aa, Artificial Sequence
  Flexible linker
AAAGSGGASAS

SEQ ID NO: 51 - protein, 16 aa, Artificial Sequence
  Flexible linker
  misc_feature (7)..(7): Xaa can be any naturally occurring amino acid
AAAGSGXGGGSGASAS

SEQ ID NO: 52 - protein, 21 aa, Artificial Sequence
  Flexible linker
  misc_feature (7)..(7): Xaa can be any naturally occurring amino acid
  misc_feature (12)..(12): Xaa can be any naturally occurring amino acid
AAAGSGXGGGSXGGGSGASAS

SEQ ID NO: 53 - protein, 26 aa, Artificial Sequence
  Flexible linker
  misc_feature (7)..(7): Xaa can be any naturally occurring amino acid
  misc_feature (12)..(12): Xaa can be any naturally occurring amino acid
  misc_feature (17)..(17): Xaa can be any naturally occurring amino acid
AAAGSGXGGGSXGGGSXGGGSGASAS

SEQ ID NO: 54 - protein, 31 aa, Artificial Sequence
  Flexible linker
  misc_feature (7)..(7): Xaa can be any naturally occurring amino acid
  misc_feature (12)..(12): Xaa can be any naturally occurring amino acid
  misc_feature (17)..(17): Xaa can be any naturally occurring amino acid
  misc_feature (22)..(22): Xaa can be any naturally occurring amino acid
AAAGSGXGGGSXGGGSXGGGSXGGGSGASAS

SEQ ID NO: 55 - protein, 13 aa, Artificial Sequence
  Flexible linker
  misc_feature (7)..(7): Xaa can be any naturally occurring amino acid
AAAGSGXSGASAS

SEQ ID NO: 56 - protein, 28 aa, Artificial Sequence
  Flexible linker
PGGNRGTTTTRRPATTTGSSPGPTQSHY

SEQ ID NO: 57 - protein, 11 aa, Artificial Sequence
  Flexible linker
ATTTGSSPGPT

SEQ ID NO: 58 - protein, 6 aa, Artificial Sequence
  Flexible linker
ATTTGS

SEQ ID NO: 59 - protein, 21 aa, Artificial Sequence
  Flexible linker
EPSGPISTINSPPSKESHKSP

SEQ ID NO: 60 - protein, 15 aa, Artificial Sequence
  Flexible linker
GTVAAPSVFIFPPSD

SEQ ID NO: 61 - protein, 15 aa, Artificial Sequence
  Flexible linker
GGGGIAPSMVGGGGS

SEQ ID NO: 62 - protein, 15 aa, Artificial Sequence
  Flexible linker
GGGGKVEGAGGGGGS

SEQ ID NO: 63 - protein, 15 aa, Artificial Sequence
  Flexible linker
GGGGSMKSHDGGGGS

SEQ ID NO: 64 - protein, 15 aa, Artificial Sequence
  Flexible linker
GGGGNLITIVGGGGS

SEQ ID NO: 65 - protein, 15 aa, Artificial Sequence
  Flexible linker
GGGGVVPSLPGGGGS

SEQ ID NO: 66 - protein, 12 aa, Artificial Sequence
  Flexible linker
GGEKSIPGGGGS

SEQ ID NO: 67 - protein, 18 aa, Artificial Sequence
  Flexible linker
RPLSYRPPFPFGFPSVRP

SEQ ID NO: 68 - protein, 18 aa, Artificial Sequence
  Flexible linker
YPRSIYIRRRHPSPSLTT

SEQ ID NO: 69 - protein, 18 aa, Artificial Sequence
  Flexible linker
TPSHLSHILPSFGLPTFN

SEQ ID NO: 70 - protein, 18 aa, Artificial Sequence
  Flexible linker
RPVSPFTFPRLSNSWLPA

SEQ ID NO: 71 - protein, 18 aa, Artificial Sequence
  Flexible linker
SPAAHFPRSIPRPGPIRT

SEQ ID NO: 72 - protein, 18 aa, Artificial Sequence
  Flexible linker
APGPSAPSHRSLPSRAFG

SEQ ID NO: 73 - protein, 18 aa, Artificial Sequence
  Flexible linker
PRNSIHFLHPLLVAPLGA

SEQ ID NO: 74 - protein, 18 aa, Artificial Sequence
  Flexible linker
MPSLSGVLQVRYLSPPDL

SEQ ID NO: 75 - protein, 18 aa, Artificial Sequence
  Flexible linker
SPQYPSPLTLTLPPHPSL

SEQ ID NO: 76 - protein, 18 aa, Artificial Sequence
  Flexible linker
NPSLNPPSYLHRAPSRIS

SEQ ID NO: 77 - protein, 17 aa, Artificial Sequence
  Flexible linker
LPWRTSLLPSLPLRRRP

SEQ ID NO: 78 - protein, 18 aa, Artificial Sequence
  Flexible linker
PPLFAKGPVGLLSRSFPP

SEQ ID NO: 79 - protein, 18 aa, Artificial Sequence
  Flexible linker
VPPAPVVSLRSAHARPPY

SEQ ID NO: 80 - protein, 17 aa, Artificial Sequence
  Flexible linker
LRPTPPRVRSYTCCPTP

SEQ ID NO: 81 - protein, 18 aa, Artificial Sequence
  Flexible linker
PNVAHVLPLLTVPWDNLR

SEQ ID NO: 82 - protein, 18 aa, Artificial Sequence
  Flexible linker
CNPLLPLCARSPAVRTFP

SEQ ID NO: 83 - protein, 11 aa, Artificial Sequence
  peptide sequence
GAPAPAAPAPA

SEQ ID NO: 84 - protein, 4 aa, Artificial Sequence
  peptide sequence
PPPP

SEQ ID NO: 85 - protein, 10 aa, Artificial Sequence
  CDRH1 sequence of antibody 645
GIDLSNYAIN

SEQ ID NO: 86 - protein, 16 aa, Artificial Sequence
  CDRH2 sequence of antibody 645
IIWASGTTFYATWAKG

SEQ ID NO: 87 - protein, 13 aa, Artificial Sequence
  CDRH3 sequences of antibody 645
TVPGYSTAPYFDL

SEQ ID NO: 88 - protein, 12 aa, Artificial Sequence
  CDRL1 sequence of antibody 645
QSSPSVWSNFLS

SEQ ID NO: 89 - protein, 7 aa, Artificial Sequence
  CDRL2 sequences of antibody 645
EASKLTS

SEQ ID NO: 90 - protein, 11 aa, Artificial Sequence
  CDRL3 sequence of antibody 645
GGGYSSISDTT

SEQ ID NO: 91 - protein, 112 aa, Artificial Sequence [sequence omitted]

SEQ ID NO: 92 - protein, 219 aa, Artificial Sequence
  1519 gL20 light chain (V + constant)
DIQMTQSPSSLSASVGDRVTITCKSSQSLVGASGKTYLYWLFQKPGKAPKRLIYLVSTLDSGIPSRFSGSGSGTEFTLTISSLQPEDFATYYCLQGTHFPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 117 aa, Artificial Sequence [sequence omitted]

SEQ ID NO: 94 - protein, 220 aa, Artificial Sequence
  1519gH20 Fab heavy chain (V + human gamma-1 CH1 no hinge)
EVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

(19) United States
(12) Patent Application Publication
(10) Pub. No.: US 2018/0127498 A1
(43) Pub. Date: May 10, 2018

(54) ANTI-FCRN ANTIBODIES

(71) Applicant: UCB Biopharma SPRL, Brussels (BE)

(72) Inventors: Pallavi BHATTA, Slough, Berkshire (GB); Emma DAVE, Slough, Berkshire (GB); Sam Philip HEYWOOD, Slough, Berkshire (GB); David Paul HUMPHREYS, Slough, Berkshire (GB); Bryan John SMITH, Slough, Berkshire (GB)

(73) Assignee: UCB Biopharma SPRL, Brussels (BE)

(21) Appl. No.: 15/573,185

(22) PCT Filed: May 9, 2016
(86) PCT No.: PCT/EP2016/060305

§ 371 Date: Nov. 10, 2017

(30) Foreign Application Priority Data
May 13, 2015 (GB) 1508180.5

(51) Int. Cl. C07K 16/28; C07K 16/18; A61P 21/04; A61P 17/00; A61P 25/00; A61P 37/00; A61P 7/00

(52) U.S. Cl. CPC C07K 16/283; C07K 16/18; A61P 21/04; A61P 17/00; A61P 25/00; A61P 37/00; A61K 2039/505; C07K 2319/74; C07K 2317/55; C07K 2317/622; C07K 2317/565; C07K 2317/56; C07K 2317/624; A61P 7/00

(57) ABSTRACT

The disclosure relates to antibody fusion proteins specific to FcRn, formulations comprising the same, use of each in therapy, processes for expressing and optionally formulating said antibody, DNA encoding the antibodies and hosts comprising said DNA.

The disclosure relates to antibodies specific to FcRn, formulations comprising the same, use of each in therapy, processes for expressing and optionally formulating said antibody, DNA encoding the antibodies and hosts comprising said DNA.

FcRn is a non-covalent complex of membrane protein FcRn a chain and β2 microglobulin (β2M). In adult mammals FcRn plays a key role in maintaining serum antibody levels by acting as a receptor that binds and salvages antibodies of the IgG isotype. IgG molecules are endocytosed by endothelial cells, and if they bind to FcRn, are recycled transcytosed out into, for example circulation. In contrast, IgG molecules that do not bind to FcRn enter the cells and are targeted to the lysosomal pathway where they are degraded. A variant IgG1 in which His435 is mutated to alanine results in the selective loss of FcRn binding and a significantly reduced serum half-life (Firan et al. 2001, International Immunology 13:993).

It is hypothesised that FcRn is a potential therapeutic target for certain autoimmune disorders caused at least in part by autoantibodies. The current treatment for certain such disorders includes plasmapheresis. Sometimes the plasmapheresis is employed along with immunosuppressive therapy for long-term management of the disease. Plasma exchange offers the quickest short-term answer to removing harmful autoantibodies. However, it may also be desirable to suppress the production of autoantibodies by the immune system, for example by the use of medications such as prednisone, cyclophosphamide, cyclosporine, mycophenolate mofetil, rituximab or a mixture of these.

Examples of diseases that can be treated with plasmapheresis include: Guillain-Barré syndrome; Chronic inflammatory demyelinating polyneuropathy; Goodpasture's syndrome; hyperviscosity syndromes; cryoglobulinemia; paraproteinemia; Waldenström macroglobulinemia; myasthenia gravis; thrombotic thrombocytopenic purpura (TTP)/hemolytic uremic syndrome; Wegener's granulomatosis; Lambert-Eaton Syndrome; antiphospholipid antibody syndrome (APS or APLS); microscopic polyangiitis; recurrent focal and segmental glomerulosclerosis in the transplanted kidney; HELLP syndrome; PANDAS syndrome; Refsum disease; Behcet syndrome; HIV-related neuropathy; Graves' disease in infants and neonates; pemphigus vulgaris; multiple sclerosis, rhabdomyolysis and alloimune diseases.

Plasmapheresis is sometimes used as a rescue therapy for removal of Fc containing therapeutics, for example in emergencies to reduced serious side effects.

Though plasmapheresis is helpful in certain medical conditions there are potential risks and complications associated with the therapy. Insertion of a rather large intravenous catheter can lead to bleeding, lung puncture (depending on the site of catheter insertion), and, if the catheter is left in too long, it can lead to infection and/or damage to the veins giving limited opportunity to repeat the procedure.

The procedure has further complications associated with it, for example when a patient's blood is outside of the body passing through the plasmapheresis instrument, the blood has a tendency to clot. To reduce this tendency, in one common protocol, citrate is infused while the blood is running through the circuit. Citrate binds to calcium in the blood, calcium being essential for blood to clot. Citrate is very effective in preventing blood from clotting; however, its use can lead to life-threateningly low calcium levels. This can be detected using the Chvostek's sign or Trousseau's sign. To prevent this complication, calcium is infused intravenously while the patient is undergoing the plasmapheresis; in addition, calcium supplementation by mouth may also be given.

Other complications of the procedure include: hypotension; potential exposure to blood products, with risk of transfusion reactions or transfusion transmitted diseases, suppression of the patient's immune system and bleeding or hematoma from needle placement.

Additionally facilities that provide plasmapheresis are limited and the procedure is very expensive.

An alternative to plasmapheresis is intravenous immunoglobulin (IVIG), which is a blood product containing pooled polyclonal IgG extracted from the plasma of over one thousand blood donors. The therapy is administered intravenously and lasts in the region of 2 weeks to 3 months.

Complications of the IVIG treatment include headaches, dermatitis, viral infection from contamination of the therapeutic product, for example HIV or hepatitis, pulmonary edema, allergic reactions, acute renal failure, venous thrombosis and aseptic meningitis.

Thus there is a significant unmet need for therapies for autoimmune disorders which are less invasive and which expose the patients to less medical complications.

Thus there is a significant unmet need for therapies for immunological disorders and/or autoimmune disorders which are less invasive and which expose the patients to less medical complications.

Accordingly agents that block or reduce the binding of IgG to FcRn may be useful in the treatment or prevention of such autoimmune and inflammatory diseases. Anti-FcRn antibodies have been described previously in WO2009/131702, WO2007/087289, WO2006/118772, WO2014/019727 and WO2014/204280.

### SUMMARY OF THE DISCLOSURE

The present invention provides an anti-FcRn antibody fusion protein comprising a Fab fragment linked directly or via a linker to a scFv wherein the Fab fragment binds to FcRn and the scFv binds to a serum carrier protein, such as albumin. In one example the scFv is a disulphide stabilised scFv (dsscFv).
Thus in one aspect there is provided an anti-FcRn antibody fusion protein comprising a Fab fragment linked directly or via a linker to a scFv wherein the Fab fragment comprises a heavy chain and a light chain wherein the variable region of the heavy chain comprises three CDRs, wherein CDR H1 has the sequence given in SEQ ID NO: 1, CDR H2 has the sequence given in SEQ ID NO: 2, and CDR H3 has the sequence given in SEQ ID NO: 3 and the variable region of the light chain comprises three CDRs, wherein CDR L1 has the sequence given in SEQ ID NO: 4, CDR L2 has the sequence given in SEQ ID NO: 5 or SEQ ID NO: 7 and CDR L3 has the sequence given in SEQ ID NO: 6.

In particular there is provided an anti-FcRn antibody fusion protein in which the scFv or dsscFv binds albumin and comprises a heavy chain variable domain comprising three CDRs, wherein CDR H1 has the sequence given in SEQ ID NO: 85, CDR H2 has the sequence given in SEQ ID NO: 86, and CDR H3 has the sequence given in SEQ ID NO: 87 and a light chain variable domain comprising three CDRs, wherein CDR L1 has the sequence given in SEQ ID NO: 88, CDR L2 has the sequence given in SEQ ID NO: 89 or SEQ ID NO: 7 and CDR L3 has the sequence given in SEQ ID NO: 90.

In particular there is provided an anti-FcRn antibody fusion protein in which the scFv is disulphide stabilised, said fusion protein comprising a heavy chain having the sequence given in SEQ ID NO:12 and a light chain having the sequence given in SEQ ID NO:10.

The antibodies of the disclosure block binding of IgG to FcRn and are thought to be useful in reducing one or more biological functions of FcRn, including reducing half-life of circulating antibodies. This may be beneficial in that it allows the patient to more rapidly clear antibodies, such as autoantibodies. Accordingly antibodies of the disclosure reduce binding of IgG to FcRn.

Importantly the antibodies of the present invention are able to bind human FcRn, for example at both pH6 and pH7.4 with comparable and high binding affinity. Advantageously therefore the antibodies are able to continue to bind FcRn even within the endosome, thereby maximising the blocking of FcRn binding to IgG.

In one embodiment antibodies and binding fragments of the present disclosure block binding of human IgG to human FcRn.

In one embodiment antibodies and binding fragments of the present disclosure do not bind β2 microglobulin.

In one embodiment antibodies and binding fragments of the present disclosure do not bind human β2 microglobulin

In one example antibodies and binding fragments of the present disclosure do not reduce circulating albumin levels by more than 50%, preferably by no more than 25%.

In one example antibodies and binding fragments of the present disclosure do not reduce circulating albumin levels.

The disclosure also extends to a polynucleotide, such as DNA, encoding an antibody or fragment as described herein, for example where the DNA is incorporated into a vector.

Also provided is a host cell comprising said polynucleotide. Methods of expressing an antibody or fragment are provided herein as are methods of conjugating an antibody or fragment to a polymer, such as PEG.

The present disclosure also relates to pharmaceutical compositions comprising said antibodies and fragments.

In one embodiment there is provided a method of treatment comprising administering a therapeutically effective amount of an antibody, fragment or composition as described herein. The present disclosure also extends to an antibody, fragment or composition according to the present disclosure for use in treatment, particularly in the treatment of an immunological and/or autoimmune disorder.

Thus the present disclosure provides antibodies, fragments thereof and methods for removal of pathogenic IgG, which is achieved by accelerating the body's natural mechanism for catabolising IgG.

In essence the antibodies and fragments according to the disclosure block the system that recycles IgG in the body.

The present therapy is likely to provide a replacement or supplement for certain diseases where plasmapheresis is a therapy or IVIg therapy, which is advantageous for patients.

### BRIEF DESCRIPTION OF THE FIGURES

FIG. 1 shows 1735h5.hFab-scFv.768 inhibits IgG recycling in MDCK II clone 7 cells

FIG. 2 shows the effect of 1735h5.hFab-scFv.768 on the concentration of human IVIg in serum of human FcRn-trangenic mice

FIG. 3 shows the effect of 1735h5.hFab-scFv.768 on the concentration of serum albumin in human FcRn-transgenic mice.

FIG. 4 shows the pharmacokinetics of 1735h5.hFab-scFv.768in normal mice.

FIG. 5 shows the pharmacokinetics of 1735h5.hFab-scFv.768in human FcRn-transgenic mice.

FIG. 6 shows thermal stability of 1735h5.hFab-scFv.768 (Fab-scFv) compared with parent Fab and equivalent Fab-Fv.

FIG. 7 shows Fab-scFv and Fab-dsscFv fragment formats of the present disclosure

FIG. 8 Antibody sequences according to the present disclosure

FIG. 9a Humanisation of antibody 1638.g49

FIG. 9b Humanisation of antibody 1638.g49

### DETAILS OF THE DISCLOSURE

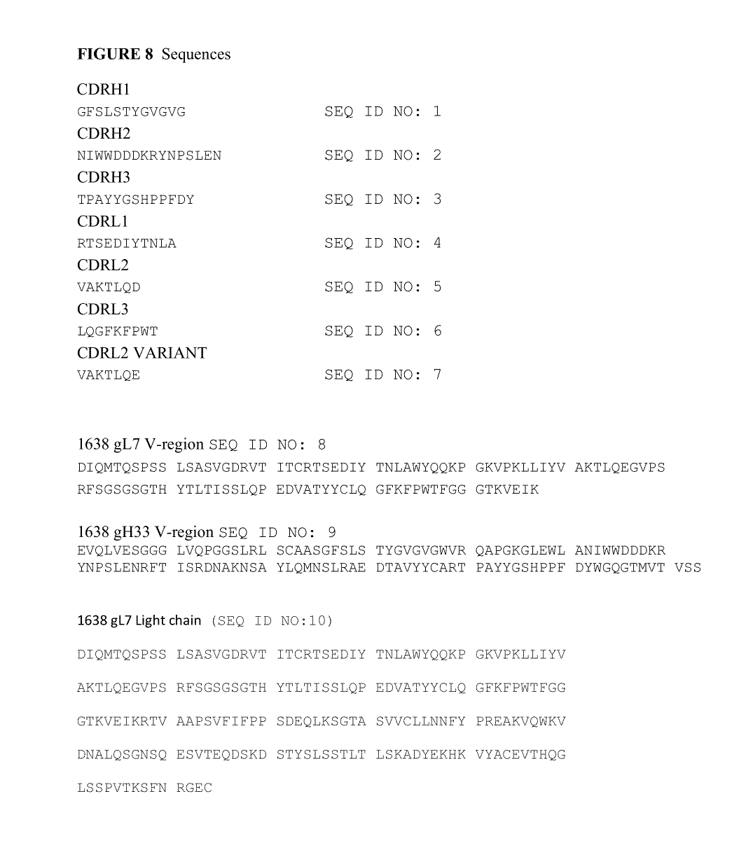

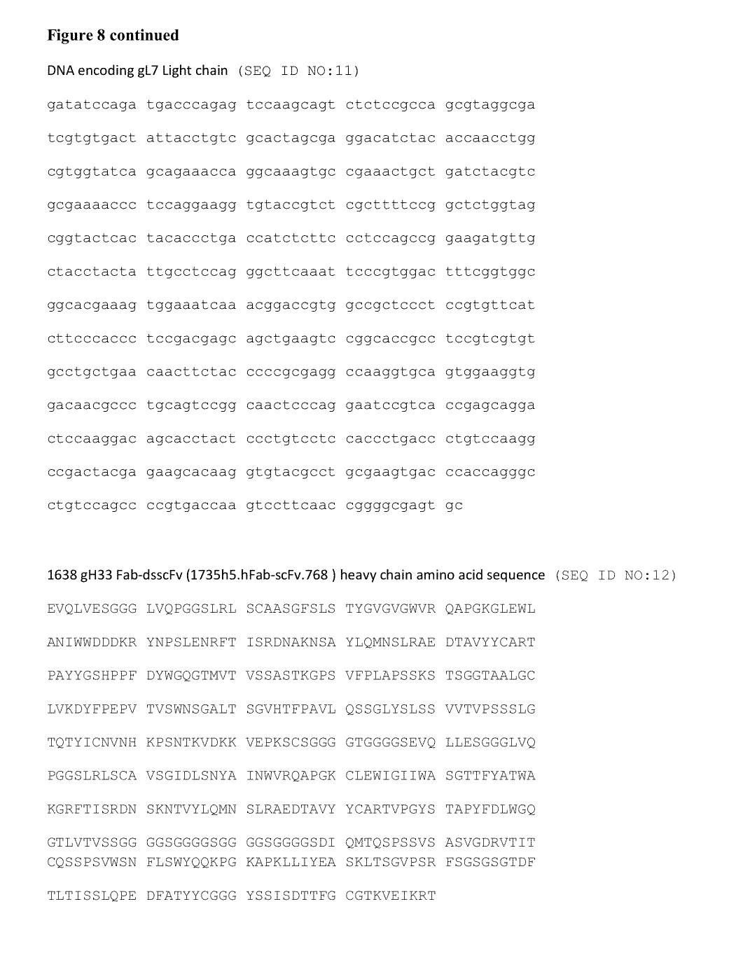

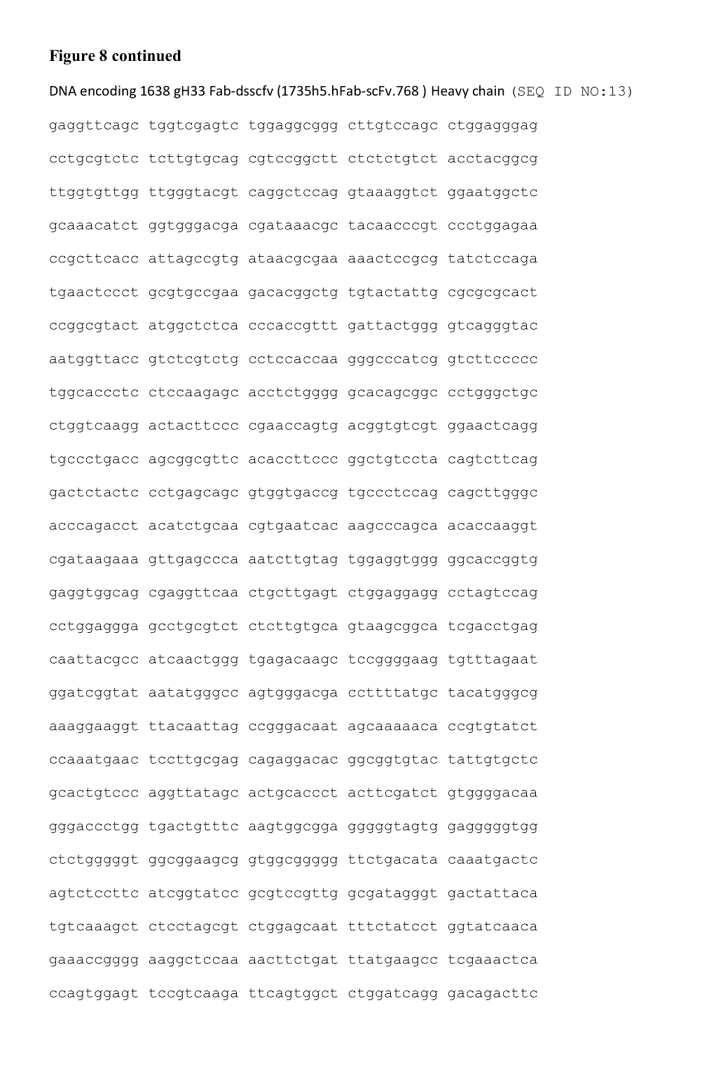

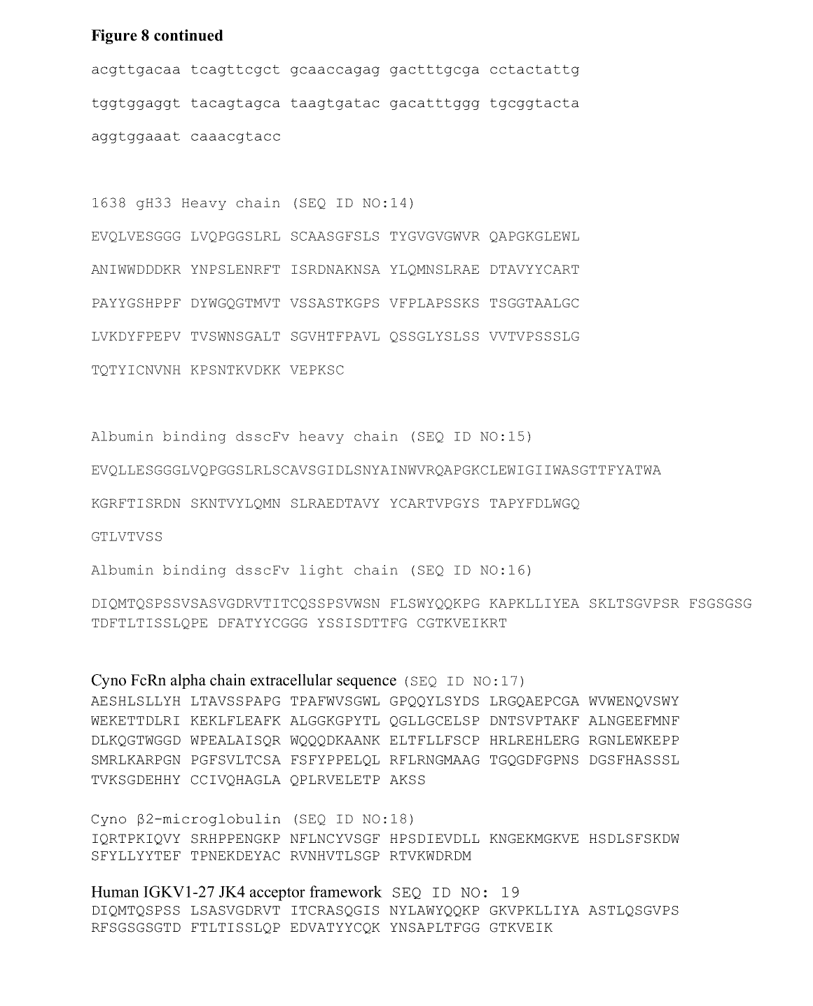

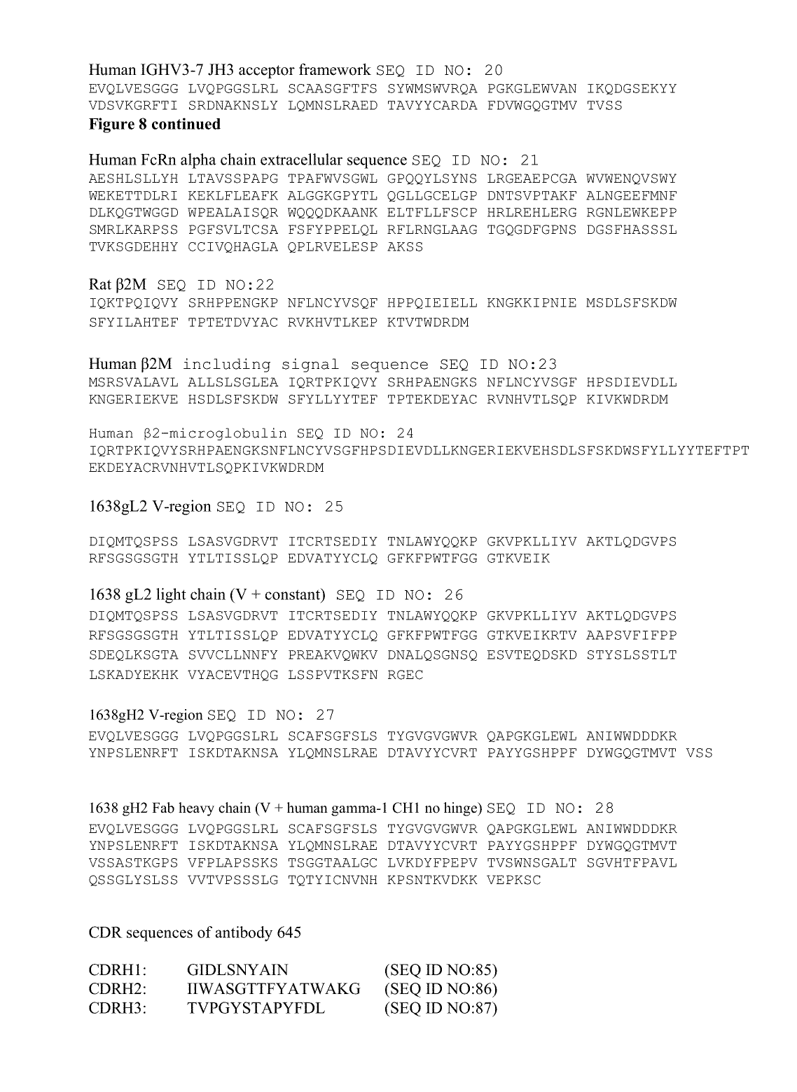

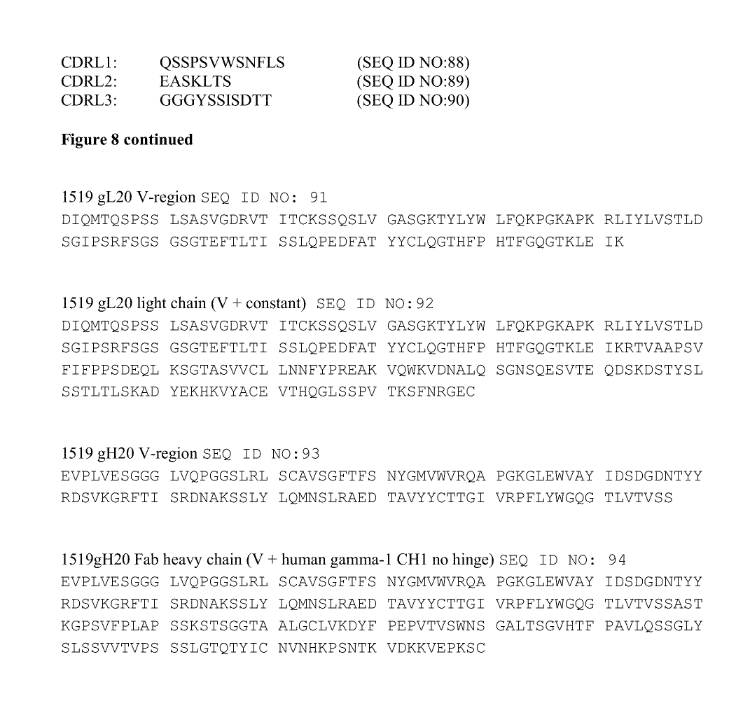

FcRn as employed herein refers to the non-covalent complex between the human IgG receptor alpha chain, also known as the neonatal Fc receptor, the amino acid sequence of which is in UniProt under number P55899, the extracellular domain of which is provided in FIG. 8 (SEQ ID NO:21), together with human β2 microglobulin (β2M), the amino acid sequence of which is in UniProt under number P61769 (provided herein with signal peptide (SEQ ID NO:23), without signal peptide (SEQ ID NO:24)).

Antibody molecule as employed herein refers to an antibody or binding fragment thereof, which includes antibody fusion proteins.

The term ‘antibody’ as used herein generally relates to intact (whole) antibodies i.e. comprising the elements of two heavy chains and two light chains. The antibody may comprise further additional binding domains, for example as per the molecule DVD-Ig as disclosed in WO 2007/024715, or the so-called (FabFv)₂Fc described in WO2011/030107. Thus antibody as employed herein includes bi, tri or tetra-valent full length antibodies.

Binding fragments of antibodies include single chain antibodies (i.e. a full length heavy chain and light chain); Fab, modified Fab, Fab′, modified Fab′, F(ab′)₂, Fv, Fab-Fv, Fab-dsFv, single domain antibodies (e.g. VH or VL or VHH), scFv, dsscFv, bi, tri or tetra-valent antibodies, Bis-scFv, diabodies, tribodies, triabodies, tetrabodies and epitope-binding fragments of any of the above (see for example Holliger and Hudson, 2005, Nature Biotech. 23(9):1126-1136; Adair and Lawson, 2005, Drug Design Reviews—Online 2(3), 209-217). The methods for creating and manufacturing these antibody fragments are well known in the art (see for example Verma et al., 1998, Journal of Immunological Methods, 216, 165-181). The Fab-Fv format was first disclosed in WO2009/040562 and the disulphide stabilised versions thereof, the Fab-dsFv was first disclosed in WO2010/035012. Other antibody fragments for use in the present invention include the Fab and Fab′ fragments described in International patent applications WO2005/003169, WO2005/003170 and WO2005/003171. Multi-valent antibodies may comprise multiple specificities e.g. bispecific or may be monospecific (see for example WO92/22583 and WO05/113605). One such example of the latter is a Tri-Fab (or TFM) as described in WO92/22583.

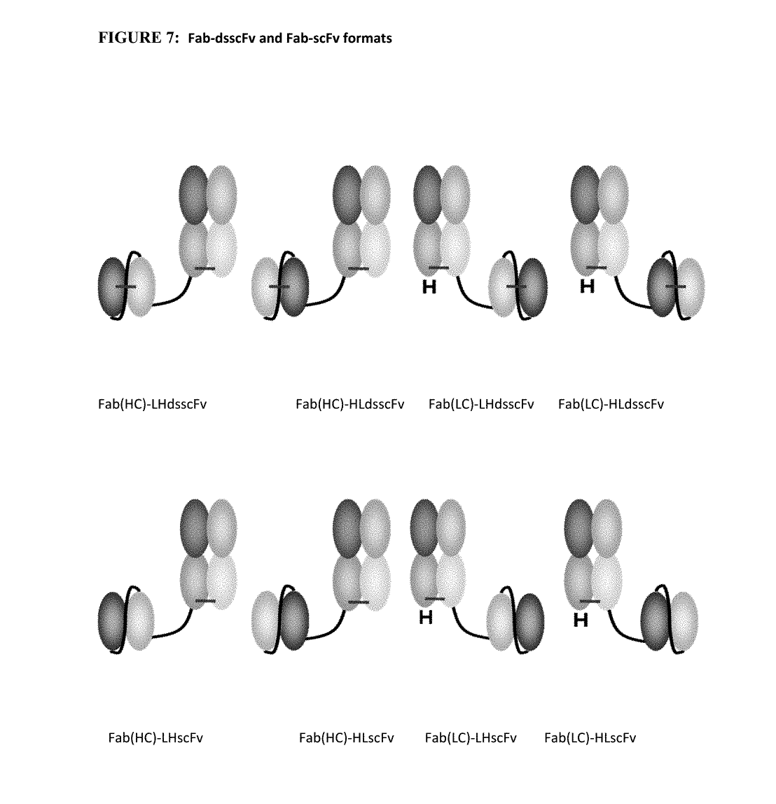

Antibody fragments also include Fab-scFv fragments and Fab-dsscFv fragments, for example as described in Example 4 of WO2013/068571 and in the present Examples and as illustrated in FIG. 7 herein. Such fragments are also termed herein ‘antibody fusion proteins’.

In one example the present invention provides an anti-FcRn antibody fusion protein comprising a Fab fragment linked directly or via a linker to a dsscFv wherein the Fab fragment binds to FcRn and the dsscFv binds to a serum carrier protein, such as albumin. Accordingly, the antibody fusion proteins of the present invention are monovalent for FcRn. It will be appreciated that the Fab fragment may be derived from any suitable anti-FcRn antibody molecule and the dsscFv may be derived from any suitable albumin binding antibody molecule.

Thus in one aspect there is provided an anti-FcRn Fab-scFv or Fab-dsscFv antibody fragment or antibody fusion protein comprising a Fab fragment linked directly or via a linker to a scFv wherein the Fab fragment comprises a heavy chain and a light chain wherein the variable region of the heavy chain comprises three CDRs, wherein CDR H1 has the sequence given in SEQ ID NO: 1, CDR H2 has the sequence given in SEQ ID NO: 2, and CDR H3 has the sequence given in SEQ ID NO: 3 and the variable region of the light chain comprises three CDRs, wherein CDR L1 has the sequence given in SEQ ID NO: 4, CDR L2 has the sequence given in SEQ ID NO: 5 or SEQ ID NO: 7 and CDR L3 has the sequence given in SEQ ID NO: 6

A typical Fab molecule for use in the present invention comprises a heavy and a light chain pair in which the heavy chain comprises a variable region V_{H} and a constant domain C_{H1}, which may terminate at the interchain cysteine and the light chain comprises a variable region V_{L} and a constant domain C_{L}.

It will be appreciated that one or more (for example 1, 2, 3 or 4) amino acid substitutions, additions and/or deletions may be made to the CDRs or other sequences (e.g variable domains) provided by the present invention without significantly altering the ability of the antibody to bind to FcRn. The effect of any amino acid substitutions, additions and/or deletions can be readily tested by one skilled in the art, for example by using the methods described herein, in particular in the Examples, to determine FcRn binding/blocking.

In one example, one or more (for example 1, 2, 3 or 4) amino acid substitutions, additions and/or deletions may be made to the framework region employed in the antibody or fragment provided by the present invention and wherein binding affinity to FcRn is retained or increased.

The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al. (supra)”). This numbering system is used in the present specification except where otherwise indicated.

The Kabat residue designations do not always correspond directly with the linear numbering of the amino acid residues. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence.

The CDRs of the heavy chain variable domain are located at residues 31-35 (CDR-H1), residues 50-65 (CDR-H2) and residues 95-102 (CDR-H3) according to the Kabat numbering system. However, according to Chothia (Chothia, C. and Lesk, A. M. J. Mol. Biol., 196, 901-917 (1987)), the loop equivalent to CDR-H1 extends from residue 26 to residue 32. Thus unless indicated otherwise ‘CDR-H1’ as employed herein is intended to refer to residues 26 to 35, as described by a combination of the Kabat numbering system and Chothia's topological loop definition.

The CDRs of the light chain variable domain are located at residues 24-34 (CDR-L1), residues 50-56 (CDR-L2) and residues 89-97 (CDR-L3) according to the Kabat numbering system.

Antibodies and fragments of the present disclosure block FcRn and may thereby prevent it functioning in the recycling of IgG. Blocking as employed herein refers to physically blocking such as occluding the receptor but will also include where the antibody or fragments binds an epitope that causes, for example a conformational change which means that the natural ligand to the receptor no longer binds. Antibody molecules of the present invention bind to FcRn and thereby decrease or prevent (e.g. inhibit) FcRn binding to an IgG constant region. In one embodiment the antibody or fragment thereof binds FcRn competitively with respect to IgG.

In one example the antibody fusion protein of the invention functions as a competitive inhibitor of human FcRn binding to human IgG. In one example the antibody fusion protein binds to the IgG binding site on FcRn. In one example the antibody fusion protein blocks the IgG binding site. In one example the antibody fusion protein does not bind β2M.

Antibodies for use in the present disclosure may be obtained using any suitable method known in the art. The FcRn polypeptide/protein including fusion proteins, cells (recombinantly or naturally) expressing the polypeptide (such as fibroblasts) can be used to produce antibodies which specifically recognise FcRn, alone or incombination with 132M. The polypeptide may be the ‘mature’ polypeptide or a biologically active fragment or derivative thereof. The human protein is registered in Swiss-Prot under the number P55899. The extracellular domain of human FcRn alpha chain is provided in SEQ ID NO: 21. The sequence of mature human β2M is provided in SEQ ID NO: 24.

In one embodiment the antigen is a mutant form of FcRn which is engineered to present FcRn on the surface of a cell, such that there is little or no dynamic processing where the FcRn is internalised in the cell, for example this can be achieved by making a mutation in the cytoplasmic tail of the FcRn alpha chain, wherein di-leucine is mutated to di-alanine as described in Ober et al 2001 Int. Immunol. 13, 1551-1559.

Polypeptides, for use to immunize a host, may be prepared by processes well known in the art from genetically engineered host cells comprising expression systems or they may be recovered from natural biological sources. In the present application, the term “polypeptides” includes peptides, polypeptides and proteins. These are used interchangeably unless otherwise specified. The FcRn polypeptide may in some instances be part of a larger protein such as a fusion protein for example fused to an affinity tag or similar.

Antibodies generated against the FcRn polypeptide may be obtained, where immunisation of an animal is necessary, by administering the polypeptides to an animal, preferably a non-human animal, using well-known and routine protocols, see for example Handbook of Experimental Immunology, D. M. Weir (ed.), Vol 4, Blackwell Scientific Publishers, Oxford, England, 1986). Many warm-blooded animals, such as rabbits, mice, rats, sheep, cows, camels or pigs may be immunized. However, mice, rabbits, pigs and rats are generally most suitable.

Monoclonal antibodies may be prepared by any method known in the art such as the hybridoma technique (Kohler & Milstein, 1975, Nature, 256:495-497), the trioma technique, the human B-cell hybridoma technique (Kozbor et al., 1983, Immunology Today, 4:72) and the EBV-hybridoma technique (Cole et al., Monoclonal Antibodies and Cancer Therapy, pp 77-96, Alan R Liss, Inc., 1985).

Antibodies for use in the invention may also be generated using single lymphocyte antibody methods by cloning and expressing immunoglobulin variable region cDNAs generated from single lymphocytes selected for the production of specific antibodies by, for example, the methods described by Babcook, J. et al., 1996, Proc. Natl. Acad. Sci. USA 93(15):7843-78481; WO92/02551; WO2004/051268 and International Patent Application number WO2004/106377.

Screening for antibodies can be performed using assays to measure binding to human FcRn and/or assays to measure the ability to block IgG binding to the receptor. An example of a binding assay is an ELISA, in particular, using a fusion protein of human FcRn and human Fc, which is immobilized on plates, and employing a secondary antibody to detect anti-FcRn antibody bound to the fusion protein. Examples of suitable antagonistic and blocking assays are described herein below.

Specific as employed herein is intended to refer to an antibody that only recognises the antigen to which it is specific or an antibody that has significantly higher binding affinity to the antigen to which it is specific compared to binding to antigens to which it is non-specific, for example at least 5, 6, 7, 8, 9, 10 times higher binding affinity. Binding affinity may be measured by techniques such as BIAcore as described herein below. In one example the antibody of the present invention does not bind β2 microglobulin (β2M). In one example the antibody of the present invention binds cynomolgus FcRn. In one example the antibody molecules of the present invention do not bind rat or mouse FcRn.

The amino acid sequences and the polynucleotide sequences of certain antibody molecules according to the present disclosure are provided and form an aspect of the invention.

In one embodiment the antibodies or binding fragments according to the present disclosure are fully human, for example prepared from a phage library or similar.

In one embodiment the antibody or fragments according to the disclosure are humanised.

Humanised antibodies (which include CDR-grafted antibodies) are antibody molecules having one or more complementarity determining regions (CDRs) from a non-human species and a framework region from a human immunoglobulin molecule (see, e.g. U.S. Pat. No. 5,585,089; WO91/09967). It will be appreciated that it may only be necessary to transfer the specificity determining residues of the CDRs rather than the entire CDR (see for example, Kashmiri et al., 2005, Methods, 36, 25-34). Humanised antibodies may optionally further comprise one or more framework residues derived from the non-human species from which the CDRs were derived. The latter are often referred to as donor residues.

Thus in one embodiment as used herein, the term ‘humanised antibody molecule’ refers to an antibody molecule wherein the heavy and/or light chain contains one or more CDRs (including, if desired, one or more modified CDRs) from a donor antibody (e.g. a non-human antibody such as a murine monoclonal antibody) grafted into a heavy and/or light chain variable region framework of an acceptor antibody (e.g. a human antibody) optionally further comprising one or more framework residues derived from the non-human species from which the CDRs were derived (donor residues). For a review, see Vaughan et al, Nature Biotechnology, 16, 535-539, 1998. In one embodiment rather than the entire CDR being transferred, only one or more of the specificity determining residues from any one of the CDRs described herein above are transferred to the human antibody framework (see for example, Kashmiri et al., 2005, Methods, 36, 25-34). In one embodiment only the specificity determining residues from one or more of the CDRs described herein above are transferred to the human antibody framework. In another embodiment only the specificity determining residues from each of the CDRs described herein above are transferred to the human antibody framework.

When the CDRs or specificity determining residues are grafted, any appropriate acceptor variable region framework sequence may be used having regard to the class/type of the donor antibody from which the CDRs are derived, including mouse, primate and human framework regions.

Suitably, the humanised antibody according to the present invention has a variable domain comprising human acceptor framework regions as well as one or more of the CDRs provided specifically herein. Thus, provided in one embodiment is blocking humanised antibody which binds human FcRn wherein the variable domain comprises human acceptor framework regions and non-human donor CDRs.

Examples of human frameworks which can be used in the present invention are KOL, NEWM, REI, EU, TUR, TEI, LAY and POM (Kabat et al., supra). For example, KOL and NEWM can be used for the heavy chain, REI can be used for the light chain and EU, LAY and POM can be used for both the heavy chain and the light chain. Alternatively, human germline sequences may be used; these are available at http://www.imgt.org/

In a humanised antibody of the present invention, the acceptor heavy and light chains do not necessarily need to be derived from the same antibody and may, if desired, comprise composite chains having framework regions derived from different chains.

One such suitable framework region for the heavy chain of the humanised antibody of the present invention is derived from the human sub-group VH3 sequence IGHV3-7 together with JH3 in one example the heavy chain variable domain of the antibody comprises the sequence given in SEQ ID NO: 20.

A suitable framework region for the light chain of the humanised antibody of the present invention is derived from the human sub-group VK1 sequence IGKV1-27 sequence together with JK4 in one example the light chain variable domain of the antibody molecule comprises the sequence given in SEQ ID NO: 19.

In a humanised antibody of the present invention, the framework regions need not have exactly the same sequence as those of the acceptor antibody. For instance, unusual residues may be changed to more frequently-occurring residues for that acceptor chain class or type. Alternatively, selected residues in the acceptor framework regions may be changed so that they correspond to the residue found at the same position in the donor antibody (see Reichmann et al., 1998, Nature, 332, 323-324). Such changes should be kept to the minimum necessary to recover the affinity of the donor antibody. A protocol for selecting residues in the acceptor framework regions which may need to be changed is set forth in WO91/09967.

Thus in one embodiment 1, 2, 3, 4, or 5 residues in the framework are replaced with an alternative amino acid residue.

Accordingly, in one example there is provided a humanised antibody, wherein at least the residues at each of positions 48 and 78 of the variable domain of the heavy chain (Kabat numbering) are donor residues, see for example the sequence given in SEQ ID NO:9.

In one embodiment residue 48 of the heavy chain variable domain is replaced with an alternative amino acid, for example valine.

In one embodiment residue 78 of the heavy chain variable domain is replaced with an alternative amino acid, for example leucine.

In one embodiment residue 48 is valine and residue 78 is leucine in the humanised heavy chain variable region according to the present disclosure.

Accordingly, in one example there is provided a humanised antibody, wherein at least the residues at each of positions 70 and 71 of the variable domain of the light chain (Kabat numbering) are donor residues, see for example the sequence given in SEQ ID NO: 8.

In one embodiment residue 70 of the light chain variable domain is replaced with an alternative amino acid, for example aspartic acid.

In one embodiment residue 71 of the light chain variable domain is replaced with an alternative amino acid, for example phenylalanine.

In one embodiment residue 70 is aspartic acid and residue 71 is phenylalanine in the humanised light chain variable region according to the present disclosure.

In one embodiment the disclosure provides an antibody sequence which is 80% similar or identical to a sequence disclosed herein, for example 85%, 90%, 91%, 92%, 93%, 94%, 95% 96%, 97%, 98% or 99% over part or whole of the relevant sequence, for example a variable domain sequence, a CDR sequence or a variable domain sequence, excluding the CDRs. In one embodiment the relevant sequence is SEQ ID NO: 8 or 9. In one embodiment the relevant sequence is SEQ ID NO: 10 or 12 or 14.

In one embodiment, the present invention provides an antibody molecule which binds human FcRn comprising a heavy chain, wherein the variable domain of the heavy chain comprises a sequence having at least 80%, 85%, 90%, 91%, 92%, 93%, 94%, 95% 96%, 97%, 98% or 99% identity or similarity to a sequence herein, for example the sequence given in SEQ ID NO: 9.

In one embodiment, the present invention provides an antibody molecule which binds human FcRn comprising a light chain, wherein the variable domain of the light chain comprises a sequence having at least 80%, 85%, 90%, 91%, 92%, 93%, 94%, 95% 96%, 97%, 98% or 99% identity or similarity to the sequence given in SEQ ID NO:8.

In one embodiment the present invention provides an antibody molecule which binds human FcRn wherein the antibody has a heavy chain variable domain which is at least 90%, 91%, 92%, 93%, 94%, 95% 96%, 97%, 98% or 99% similar or identical to a sequence given herein, for example the sequence given in SEQ ID NO: 9 but wherein the antibody molecule has the sequence given in SEQ ID NO: 1 for CDR-H1, the sequence given in SEQ ID NO: 2 for CDR-H2 and the sequence given in SEQ ID NO: 3 for CDR-H3.

In one embodiment the present invention provides an antibody molecule which binds human FcRn wherein the antibody has a light chain variable domain which is at least 90%, 91%, 92%, 93%, 94%, 95% 96%, 97%, 98% or 99% similar or identical to a sequence given herein, for example the sequence in SEQ ID NO:8 but wherein the antibody molecule has the sequence given in SEQ ID NO: 4 for CDR-L1, the sequence given in SEQ ID NO: 5 or SEQ ID NO: 7 for CDR-L2 and the sequence given in SEQ ID NO: 6 for CDR-L3.

In one embodiment the present invention provides an antibody molecule which binds human FcRn wherein the antibody has a heavy chain which is at least 80%, 85%, 90% , 91%, 92%, 93%, 94%, 95% 96%, 97%, 98% or 99% similar or identical to a sequence given herein, for example the sequence given in SEQ ID NO: 12 and a light chain which is at least 90%, 91%, 92%, 93%, 94%, 95% 96%, 97%, 98% or 99% similar or identical to a sequence given herein, for example the sequence given in SEQ ID NO:10.

In one embodiment the present invention provides an antibody molecule which binds human FcRn wherein the antibody has a heavy chain which is at least 90% , 91%, 92%, 93%, 94%, 95% 96%, 97%, 98% or 99% similar or identical to a sequence given herein, for example the sequence given in SEQ ID NO: 12 and a light chain which is at least 90%, 91%, 92%, 93%, 94%, 95% 96%, 97%, 98% or 99% similar or identical to a sequence given herein, for example the sequence given in SEQ ID NO:10 but wherein the Fab portion of the antibody molecule has the sequence given in SEQ ID NO: 1 for CDR-H1, the sequence given in SEQ ID NO: 2 for CDR-H2, the sequence given in SEQ ID NO: 3 for CDR-H3, the sequence given in SEQ ID NO: 4 for CDR-L1, the sequence given in SEQ ID NO: 5 or SEQ ID NO: 7 for CDR-L2 and the sequence given in SEQ ID NO: 6 for CDR-L3.

In one embodiment the present invention provides an antibody molecule which binds human FcRn wherein the antibody has a heavy chain which is at least 90% , 91%, 92%, 93%, 94%, 95% 96%, 97%, 98% or 99% similar or identical to a sequence given herein, for example the sequence given in SEQ ID NO: 12 and a light chain which is at least 90%, 91%, 92%, 93%, 94%, 95% 96%, 97%, 98% or 99% similar or identical to a sequence given herein, for example the sequence given in SEQ ID NO:10 but wherein the dsscFv portion of the antibody molecule has the sequence given in SEQ ID NO: 85 for CDR-H1, the sequence given in SEQ ID NO: 86 for CDR-H2, the sequence given in SEQ ID NO: 87 for CDR-H3, the sequence given in SEQ ID NO: 88 for CDR-L1, the sequence given in SEQ ID NO: 89 for CDR-L2 and the sequence given in SEQ ID NO: 90 for CDR-L3.

In one embodiment the present invention provides an antibody molecule which binds human FcRn wherein the antibody has a heavy chain which is at least 90% , 91%, 92%, 93%, 94%, 95% 96%, 97%, 98% or 99% similar or identical to a sequence given herein, for example the sequence given in SEQ ID NO: 12 and a light chain which is at least 90%, 91%, 92%, 93%, 94%, 95% 96%, 97%, 98% or 99% similar or identical to a sequence given herein, for example the sequence given in SEQ ID NO:10 but wherein the Fab portion of the antibody molecule has the sequence given in SEQ ID NO: 1 for CDR-H1, the sequence given in SEQ ID NO: 2 for CDR-H2, the sequence given in SEQ ID NO: 3 for CDR-H3, the sequence given in SEQ ID NO: 4 for CDR-L1, the sequence given in SEQ ID NO: 5 or SEQ ID NO: 7 for CDR-L2 and the sequence given in SEQ ID NO: 6 for CDR-L3 and wherein the dsscFv portion of the antibody molecule has the sequence given in SEQ ID NO: 85 for CDR-H1, the sequence given in SEQ ID NO: 86 for CDR-H2, the sequence given in SEQ ID NO: 87 for CDR-H3, the sequence given in SEQ ID NO: 88 for CDR-L1, the sequence given in SEQ ID NO: 89 for CDR-L2 and the sequence given in SEQ ID NO: 90 for CDR-L3.

“Identity”, as used herein, indicates that at any particular position in the aligned sequences, the amino acid residue is identical between the sequences. “Similarity”, as used herein, indicates that, at any particular position in the aligned sequences, the amino acid residue is of a similar type between the sequences. For example, leucine may be substituted for isoleucine or valine. Other amino acids which can often be substituted for one another include but are not limited to:

phenylalanine, tyrosine and tryptophan (amino acids having aromatic side chains);
lysine, arginine and histidine (amino acids having basic side chains);
aspartate and glutamate (amino acids having acidic side chains);
asparagine and glutamine (amino acids having amide side chains); and
cysteine and methionine (amino acids having sulphur-containing side chains). Degrees of identity and similarity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987, Sequence Analysis Primer, Gribskov, M. and Devereux, J., eds., M Stockton Press, New York, 1991, the BLAST™ software available from NCBI (Altschul, S. F. et al., 1990, J. Mol. Biol. 215:403-410; Gish, W. & States, D. J. 1993, Nature Genet. 3:266-272. Madden, T. L. et al., 1996, Meth. Enzymol. 266:131-141; Altschul, S. F. et al., 1997, Nucleic Acids Res. 25:3389-3402; Zhang, J. & Madden, T. L. 1997, Genome Res. 7:649-656,).

It will be appreciated that the Fab-fragment component of the antibody fusion proteins of the present invention may be derived or constructed from any suitable anti-FcRn antibody or antibody fragment.

In one example, the antibody molecule of the present disclosure comprises an antibody Fab fragment comprising the sequence shown in SEQ ID NOs: 26 and 28, for example for the light and heavy chain respectively.

In one example, the antibody molecule of the present disclosure comprises an antibody Fab fragment comprising the sequence shown in SEQ ID NOs: 92 and 94, for example for the light and heavy chain respectively

In one example, the antibody molecule of the present disclosure comprises an antibody Fab fragment comprising the sequence shown in SEQ ID NOs: 10 and 14, for example for the light and heavy chain respectively. In one embodiment the antibody Fab fragment portion of the molecule has a light chain comprising the sequence given in SEQ ID NO: 10 and a heavy chain comprising the sequence given in SEQ ID NO: 14.

The Fab fragment of the present invention is linked, directly or via a linker to a scFv. In one example the scFv is disulphide stabilised. Typically the scFv is disulphide stabilised. The linkage to the Fab fragment can be a chemical conjugation but is most preferably a translation fusion, i.e. a genetic fusion where the sequence of each is encoded in sequence by an expression vector. The linker is therefore typically an amino acid linker as described herein.

Typically the scFv or dsscFv binds to a serum carrier protein in order to extend the half-life of the antibody fusion protein in vivo. Extending half-life in such a way is independent of FcRn binding and may be advantageous.

“Serum carrier protein” as employed herein refers to any suitable plasma carrier protein to which the scFv may bind, in one example the serum carrier protein is selected from thyroxine-binding protein, transthyretin, al-acid glycoprotein, transferrin, fibrinogen and albumin, or a fragment of any thereof

Typically the scFv or dsscFv binds to albumin, preferably human serum albumin.

Any suitable albumin binding scFv or dsscFv may be incorporated into the antibody fusion proteins of the invention. Suitable albumin binding domains have previously been described in the art.

“Single chain variable fragment” or “scFv” as employed herein refers to a single chain variable fragment which is stabilised by a peptide linker between the V_{H} and V_{L} variable domains

“Disulphide-stabilised single chain variable fragment” or “dsscFv” as employed herein refers to a single chain variable fragment which is stabilised by a peptide linker between the V_{H} and V_{L} variable domain and also includes an inter-domain disulphide bond between V_{H} and V_{L}.

In one embodiment, the disulfide bond between the variable domains V_{H} and V_{L} of the dsscFv is between two of the residues listed below (unless the context indicates otherwise Kabat numbering is employed in the list below). Wherever reference is made to Kabat numbering the relevant reference is Kabat et al., 1987, in Sequences of Proteins of Immunological Interest, US Department of Health and Human Services, NIH, USA.

In one embodiment the disulfide bond is in a position selected from the group comprising:
V_{H}37+V_{L}95C see for example Protein Science 6, 781-788 Zhu et al (1997);
V_{H}44+V_{L}100 see for example; Biochemistry 33 5451-5459 Reiter et al (1994); or Journal of Biological Chemistry Vol. 269 No. 28 pp.18327-18331 Reiter et al (1994); or Protein Engineering, vol. 10 no. 12 pp. 1453-1459 Rajagopal et al (1997);
V_{H}44+V_{L}105 see for example J Biochem. 118, 825-831 Luo et al (1995);
V_{H}45+V_{L}87 see for example Protein Science 6, 781-788 Zhu et al (1997);
V_{H}55+V_{L}101 see for example FEBS Letters 377 135-139 Young et al (1995);
V_{H}10 +V_{L}50 see for example Biochemistry 29 1362-1367 Glockshuber et al (1990);
V_{H}100b +V_{L}49;
V_{H}98+V_{L}46 see for example Protein Science 6, 781-788 Zhu et al (1997);
V_{H}101+V_{L}46;
V_{H}105+V_{L}43 see for example; Proc. Natl. Acad. Sci. USA Vol. 90 pp.7538-7542 Brinkmann et al (1993); or Proteins 19, 35-47 Jung et al (1994),
V_{H}106+V_{L}57 see for example FEBS Letters 377 135-139 Young et al (1995) and a position or positions corresponding thereto in variable region pair located in the molecule.

In one embodiment, the disulphide bond is formed between positions V_{H}44 and V_{L}100.

The amino acid pairs listed above are in the positions conducive to replacement by cysteines such that disulfide bonds can be formed. Cysteines can be engineered into these desired positions by known techniques. In one embodiment therefore an engineered cysteine according to the present disclosure refers to where the naturally occurring residue at a given amino acid position has been replaced with a cysteine residue.

Introduction of engineered cysteines can be performed using any method known in the art. These methods include, but are not limited to, PCR extension overlap mutagenesis, site-directed mutagenesis or cassette mutagenesis (see, generally, Sambrook et al., Molecular Cloning, A Laboratory Manual, Cold Spring Harbour Laboratory Press, Cold Spring Harbour, NY, 1989; Ausbel et al., Current Protocols in Molecular Biology, Greene Publishing & Wiley-Interscience, NY, 1993). Site-directed mutagenesis kits are commercially available, e.g. QuikChange® Site-Directed Mutagenesis kit (Stratagen, La Jolla, Calif.). Cassette mutagenesis can be performed based on Wells et al., 1985, Gene, 34:315-323. Alternatively, mutants can be made by total gene synthesis by annealing, ligation and PCR amplification and cloning of overlapping oligonucleotides.

Accordingly, in one embodiment the variable domains V_{H} and V_{L} of the dsscFv may be linked by a disulfide bond between two cysteine residues, wherein the position of the pair of cysteine residues is selected from the group comprising or consisting of: V_{H}37 and V_{L}95, V_{H}44 and V_{L}100, V_{H}44 and V_{L}105, V_{H}45 and V_{L}87, V_{H}100 and V_{L}50, V_{H}100b and V_{L}49, V_{H}98 and V_{L}46, V_{H}101 and V_{L}46, V_{H}105 and V_{L}43 and V_{H}106 and V_{L}57.

In one embodiment, the variable domains V_{H} and V_{L} of the dsscFv may be linked by a disulfide bond between two cysteine residues, one in V_{H} and one in V_{L}, which are outside of the CDRs, for example wherein the position of the pair of cysteine residues is selected from the group consisting of V_{H}37 and V_{L}95, V_{H}44 and V_{L}100, V_{H}44 and V_{L}105, V_{H}45 and V_{L}87, V_{H}100 and V_{L}50, V_{H}98 and V_{L}46, V_{H}105 and V_{L}43 and V_{H}106 and V_{L}57.

In one embodiment, the variable domains V_{H} and V_{L} of the dsscFv are linked by a disulphide bond between two engineered cysteine residues, one at position V_{H}44 and the other at V_{L}100.

In one embodiment, the variable domains V_{H} and V_{L} of the dsscFv are linked by a disulphide bond between two engineered cysteine residues, one at position V_{H}44 and the other at V_{L}100.

In one example the scFv or dsscFv comprises a heavy chain variable domain comprising three CDRs, wherein CDR H1 has the sequence given in SEQ ID NO: 85, CDR H2 has the sequence given in SEQ ID NO: 86, and CDR H3 has the sequence given in SEQ ID NO: 87 and a light chain variable domain comprising three CDRs, wherein CDR L1 has the sequence given in SEQ ID NO: 88, CDR L2 has the sequence given in SEQ ID NO: 89 and CDR L3 has the sequence given in SEQ ID NO: 90.

In one example the VH domain of the dsscFv of the present invention has the sequence given in SEQ ID NO:15 and the VL domain of the dsscFv of the present invention has the sequence given in SEQ ID NO:16.

The VH and VL in scFv and dsscFvs of the present invention are linked to one another via a suitable linker, typically in the Heavy-Light (HL) orientation, examples of which are provided herein below. In the antibody fusion proteins of the present invention the scFv or dsscFv is linked directly or via a linker to the C-terminus of the Fab fragment.

In one embodiment, the heavy chain variable domain of the dsscFv is attached to the C terminus of the heavy chain of the Fab fragment or the light chain variable domain of the dsscFv is attached to the C terminus of the heavy chain of the Fab fragment.

In one embodiment, the heavy chain variable domain of the dsscFv is attached to the C terminus of the light chain of the Fab fragment or the light chain variable domain of the dsscFv is attached to the C-terminus of the light chain of the Fab fragment

In one embodiment, the V_{H} domain of the dsscFv is attached via a linker to the C-terminus of CH₁ of the Fab fragment. In one embodiment, the V_{H} domain of the dsscFv is attached to the C-terminus of C_{L}.

In one embodiment the linker, is any suitable linker, for example a suitable peptide for connecting the C-terminal portion of CH₁ or C_{L} to the VH of the dsscFv.

In one embodiment a peptide linker for use in the present invention is 50 amino acids in length or less, for example 20 amino acids or less, such as 19, 10, 17, 16, 15, 14, 13, 12, 11, 10, 9, 8, 7, 6, 5, 4, 3, 2 or 1 amino acids.

In one embodiment the linker is based on repeating units of G45 (i.e. GGGGS SEQ ID NO: 29), for example 1, 2, 3, 4 or 5 units thereof, such as GGGGSGGGGSGGGGSGGGGS (SEQ ID NO:30) or SGGGGSGGGGSGGGGSGGGGS (SEQ ID NO:31). In one embodiment a linker employed in a construct of the present disclosure has the sequence SGGGGSGGGGTGGGGS (SEQ ID NO:32). In addition such linkers may be used to connect the VH and VL of the scFv or dsscFv. In one embodiment the linker is selected from a sequence shown herein. In one embodiment the VH and VL are linked by a linker having the sequence given in SEQ ID NO:30. In one embodiment the linker between the C-terminus of CH1 of the Fab and the N-terminus of the VH of the dsscFv has the sequence SGGGGTGGGGS (SEQ ID NO: 33).

TABLE 1
Hinge linker sequences

| SEQ ID NO: | SEQUENCE |
|---|---|
| 34 | DKTHTCAA |
| 35 | DKTHTCPPCPA |
| 36 | DKTHTCPPCPATCPPCPA |
| 37 | DKTHTCPPCPATCPPCPATCPPCPA |
| 38 | DKTHTCPPCPAGKPTLYNSLVMSDTAGTCY |
| 39 | DKTHTCPPCPAGKPTHVNVSVVMAEVDGTCY |
| 40 | DKTHTCCVECPPCPA |
| 41 | DKTHTCPRCPEPKSCDTPPPCPRCPA |
| 42 | DKTHTCPSCPA |

TABLE 2
Flexible linker sequences

| SEQ ID NO: | SEQUENCE |
|---|---|
| 43 | SGGGGSE |
| 44 | DKTHTS |
| 45 | (S)GGGGS |
| 46 | (S)GGGGSGGGGS |
| 47 | (S)GGGGSGGGGSGGGGS |
| 48 | (S)GGGGSGGGGSGGGGSGGGGS |
| 49 | (S)GGGGSGGGGSGGGGSGGGGSGGGGS |
| 50 | AAAGSG-GASAS |
| Si | AAAGSG-XGGGS-GASAS |
| 52 | AAAGSG-XGGGSXGGGS-GASAS |
| 53 | AAAGSG-XGGGSXGGGSXGGGS-GASAS |
| 54 | AAAGSG-XGGGSXGGGSXGGGSXGGGS-GASAS |
| 55 | AAAGSG-XS-GASAS |
| 56 | PGGNRGTTTTRRPATTTGSSPGPTQSHY |
| 57 | ATTTGSSPGPT |
| 58 | ATTTGS |
| — | GS |
| 59 | EPSGPISTINSPPSKESHKSP |
| 60 | GTVAAPSVFIFPPSD |
| 61 | GGGGIAPSMVGGGGS |
| 62 | GGGGKVEGAGGGGGS |
| 63 | GGGGSMKSHDGGGGS |
| 64 | GGGGNLITIVGGGGS |
| 65 | GGGGVVPSLPGGGGS |
| 66 | GGEKSIPGGGGS |
| 67 | RPLSYRPPFPFGFPSVRP |
| 68 | YPRSIYIRRRHPSPSLTT |
| 69 | TPSHLSHILPSFGLPTFN |
| 70 | RPVSPFTFPRLSNSWLPA |
| 71 | SPAAHFPRSIPRPGPIRT |
| 72 | APGPSAPSHRSLPSRAFG |
| 73 | PRNSIHFLHPLLVAPLGA |
| 74 | MPSLSGVLQVRYLSPPDL |
| 75 | SPQYPSPLTLTLPPHPSL |
| 76 | NPSLNPPSYLHRAPSRIS |
| 77 | LPWRTSLLPSLPLRRRP |
| 78 | PPLFAKGPVGLLSRSFPP |
| 79 | VPPAPVVSLRSAHARPPY |
| 80 | LRPTPPRVRSYTCCPTP- |
| 81 | PNVAHVLPLLTVPWDNLR |
| 82 | CNPLLPLCARSPAVRTFP |

(S) is optional in sequences 45 to 49.

Examples of rigid linkers include the peptide sequences GAPAPAAPAPA (SEQ ID NO: 83), PPPP (SEQ ID NO: 84) and PPP.

In one embodiment the Fab-dsscFv fragment or fusion protein of the present invention comprises a heavy chain comprising or consisting of the sequence given in SEQ ID NO:12 and a light chain comprising or consisting of the sequence given in SEQ ID NO:10.

CA170_01638g49 and 1638.g49 are employed inchangeably herein and are used to refer to a specific pair of antibody variable regions which may be used in a number of different formats. These variable regions are the heavy chain sequence gH33 given in SEQ ID NO: 9 and the light chain sequence gL7 given in SEQ ID NO: 8.

CA170_01638g28 and 1638.g28 are employed inchangeably herein and are used to refer to a specific pair of antibody variable regions which may be used in a number of different formats. These variable regions are the heavy chain sequence given in SEQ ID NO: 27 and the light chain sequence given in SEQ ID NO: 25.

In one embodiment the antibody Fab heavy chain comprises a CH1 domain and the antibody light chain comprises a CL domain, either kappa or lambda.

In one embodiment the light chain of the Fab component has the sequence given in SEQ ID NO: 10 and the heavy chain of the Fab component has the sequence given in SEQ ID NO: 14.

In one embodiment the light chain of the Fab component has the sequence given in SEQ ID NO: 26 and the heavy chain of the Fab component has the sequence given in SEQ ID NO: 28.

In one embodiment the light chain of the Fab component has the sequence given in SEQ ID NO: 92 and the heavy chain of the Fab component has the sequence given in SEQ ID NO: 94.

Biological molecules, such as antibodies or fragments, contain acidic and/or basic functional groups, thereby giving the molecule a net positive or negative charge. The amount of overall “observed” charge will depend on the absolute amino acid sequence of the entity, the local environment of the charged groups in the 3D structure and the environmental conditions of the molecule. The isoelectric point (pI) is the pH at which a particular molecule or solvent accessible surface thereof carries no net electrical charge. In one example, the FcRn antibody and fragments of the invention may be engineered to have an appropriate isoelectric point. This may lead to antibodies and/or fragments with more robust properties, in particular suitable solubility and/or stability profiles and/or improved purification characteristics.

Thus in one aspect the invention provides a humanised FcRn antibody engineered to have an isoelectric point different to that of the originally identified antibody. The antibody may, for example be engineered by replacing an amino acid residue such as replacing an acidic amino acid residue with one or more basic amino acid residues. Alternatively, basic amino acid residues may be introduced or acidic amino acid residues can be removed. Alternatively, if the molecule has an unacceptably high pI value acidic residues may be introduced to lower the pI, as required. It is important that when manipulating the pI care must be taken to retain the desirable activity of the antibody or fragment. Thus in one embodiment the engineered antibody or fragment has the same or substantially the same activity as the “unmodified” antibody or fragment.

Programs such as ** ExPASY http://www.expasy.ch/tools/pi_tool.html, and http://www.iut-arles.up.univ-mrs.fr/w3bb/d_abim/compo-p.html, may be used to predict the isoelectric point of the antibody or fragment. Alternatively or additionally, the pI can be measured using any suitable standard laboratory technique.

The antibody molecules of the present invention suitably have a high binding affinity, in particular in the nanomolar range. Affinity may be measured using any suitable method known in the art, including BIAcore, as described in the Examples herein, using isolated natural or recombinant FcRn or a suitable fusion protein/polypeptide. In one example affinity is measured using recombinant human FcRn extracellular domain as described in the Examples herein (SEQ ID NO: 21). In one example affinity is measured using the recombinant human FcRn alpha chain extracellular domain (SEQ ID NO: 21) in association with human β2 microglobulin (β2M) (SEQ ID NO: 24). Suitably the antibody molecules of the present invention have a binding affinity for isolated human FcRn of about 1 nM or lower. In one embodiment the antibody molecule of the present invention has a binding affinity of about 500 pM or lower (i.e. higher affinity). In one embodiment the antibody molecule of the present invention has a binding affinity of about 250 pM or lower. In one embodiment the antibody molecule of the present invention has a binding affinity of about 200 pM or lower. In one embodiment the antibody molecule of the present invention has a binding affinity of about 160 pM or lower.

In one embodiment the antibody molecules of the present invention are able to bind human FcRn at both pH6 or lower pH (in particular pH 6) and pH7.4 or higher pH (in particular pH7.4) with comparable binding affinity. Advantageously therefore the antibodies are able to continue to bind FcRn even within the endosome, thereby maximising the blocking of FcRn binding to IgG.

In one embodiment the antibody molecules of the present invention are able to bind human FcRn with a binding affinity of 200 pM or lower or 160 pM or lower when measured at pH6 and pH7.4. In one embodiment the antibodies of the present invention are able to bind human FcRn with a binding affinity of 130 pM or lower when measured at pH6 and pH7.4. In one embodiment the antibodies of the present invention are able to bind human FcRn with a binding affinity of 160 pM or lower when measured at pH6 and a binding affinity of 50 pM or lower when measured at pH7.4.

The affinity of an antibody or binding fragment of the present invention, as well as the extent to which a binding agent (such as an antibody) inhibits binding, can be determined by one of ordinary skill in the art using conventional techniques, for example those described by Scatchard et al. (Ann. KY. Acad. Sci. 51:660-672 (1949)) or by surface plasmon resonance (SPR) using systems such as BIAcore. For surface plasmon resonance, target molecules are immobilized on a solid phase and exposed to ligands in a mobile phase running along a flow cell. If ligand binding to the immobilized target occurs, the local refractive index changes, leading to a change in SPR angle, which can be monitored in real time by detecting changes in the intensity of the reflected light. The rates of change of the SPR signal can be analyzed to yield apparent rate constants for the association and dissociation phases of the binding reaction. The ratio of these values gives the apparent equilibrium constant (affinity) (see, e.g., Wolff et al, Cancer Res. 53:2560-65 (1993)).

In the present invention affinity of the test antibody molecule is typically determined using SPR as follows. The test antibody molecule is captured on the solid phase and human FcRn alpha chain extracellular domain in non-covalent complex with human β2M is run over the captured antibody in the mobile phase and affinity of the test antibody molecule for human FcRn determined. The test antibody molecule may be captured on the solid phase chip surface using any appropriate method, for example using an anti-Fc or anti Fab′ specific capture agent. In one example the affinity is determined at pH6. In one example the affinity is determined at pH7.4.

It will be appreciated that the affinity of antibodies provided by the present invention may be altered using any suitable method known in the art. The present invention therefore also relates to variants of the antibody molecules of the present invention, which have an improved affinity for FcRn. Such variants can be obtained by a number of affinity maturation protocols including mutating the CDRs (Yang et al., J. Mol. Biol., 254, 392-403, 1995), chain shuffling (Marks et al., Bio/Technology, 10, 779-783, 1992), use of mutator strains of E. coli (Low et al., J. Mol. Biol., 250, 359-368, 1996), DNA shuffling (Patten et al., Curr. Opin. Biotechnol., 8, 724-733, 1997), phage display (Thompson et al., J. Mol. Biol., 256, 77-88, 1996) and sexual PCR (Crameri et al., Nature, 391, 288-291, 1998). Vaughan et al. (supra) discusses these methods of affinity maturation.

In one embodiment the antibody molecules of the present invention block human FcRn activity. Assays suitable for determining the ability of an antibody to block FcRn are described in the Examples herein. A suitable assay for determining the ability of an antibody molecule to block IgG recycling in vitro is described herein below.

If desired an antibody molecule for use in the present invention may be conjugated to one or more effector molecule(s). It will be appreciated that the effector molecule may comprise a single effector molecule or two or more such molecules so linked as to form a single moiety that can be attached to the antibodies of the present invention. Where it is desired to obtain an antibody fragment linked to an effector molecule, this may be prepared by standard chemical or recombinant DNA procedures in which the antibody fragment is linked either directly or via a coupling agent to the effector molecule. Techniques for conjugating such effector molecules to antibodies are well known in the art (see, Hellstrom et al., Controlled Drug Delivery, 2nd Ed., Robinson et al., eds., 1987, pp. 623-53; Thorpe et al., 1982 , Immunol. Rev., 62:119-58 and Dubowchik et al., 1999, Pharmacology and Therapeutics, 83, 67-123). Particular chemical procedures include, for example, those described in WO 93/06231, WO 92/22583, WO 89/00195, WO 89/01476 and WO 03/031581. Alternatively, where the effector molecule is a protein or polypeptide the linkage may be achieved using recombinant DNA procedures, for example as described in WO 86/01533 and EP0392745.

The term effector molecule as used herein includes, for example, antineoplastic agents, drugs, toxins, biologically active proteins, for example enzymes, other antibody or antibody fragments, synthetic or naturally occurring polymers, nucleic acids and fragments thereof e.g. DNA, RNA and fragments thereof, radionuclides, particularly radioiodide, radioisotopes, chelated metals, nanoparticles and reporter groups such as fluorescent compounds or compounds which may be detected by NMR or ESR spectroscopy.

Examples of effector molecules may include cytotoxins or cytotoxic agents including any agent that is detrimental to (e.g. kills) cells. Examples include combrestatins, dolastatins, epothilones, staurosporin, maytansinoids, spongistatins, rhizoxin, halichondrins, roridins, hemiasterlins, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

Effector molecules also include, but are not limited to, antimetabolites (e.g. methotrexate, 6-mercaptopurine, 6-thioguanine, cytarabine, 5-fluorouracil decarbazine), alkylating agents (e.g. mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g. daunorubicin (formerly daunomycin) and doxorubicin), antibiotics (e.g. dactinomycin (formerly actinomycin), bleomycin, mithramycin, anthramycin (AMC), calicheamicins or duocarmycins), and anti-mitotic agents (e.g. vincristine and vinblastine).

Other effector molecules may include chelated radionuclides such as ¹¹¹In and ⁹⁰Y, Lu¹⁷⁷, Bismuth²¹³, Californium²⁵², Iridium¹⁹² and Tungsten¹⁸⁸/Rhenium¹⁸⁸; or drugs such as but not limited to, alkylphosphocholines, topoisomerase I inhibitors, taxoids and suramin. Other effector molecules include proteins, peptides and enzymes. Enzymes of interest include, but are not limited to, proteolytic enzymes, hydrolases, lyases, isomerases, transferases. Proteins, polypeptides and peptides of interest include, but are not limited to, immunoglobulins, toxins such as abrin, ricin A, pseudomonas exotoxin, or diphtheria toxin, a protein such as insulin, tumour necrosis factor, α-interferon, β-interferon, nerve growth factor, platelet derived growth factor or tissue plasminogen activator, a thrombotic agent or an anti-angiogenic agent, e.g. angiostatin or endostatin, or, a biological response modifier such as a lymphokine, interleukin-1 (IL-1), interleukin-2 (IL-2), granulocyte macrophage colony stimulating factor (GM-CSF), granulocyte colony stimulating factor (G-CSF), nerve growth factor (NGF) or other growth factor and immunoglobulins.

Other effector molecules may include detectable substances useful for example in diagnosis. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include ¹²⁵I, ¹³¹I, ¹¹¹In and ⁹⁹Tc.

In another example the effector molecule may increase the half-life of the antibody in vivo, and/or reduce immunogenicity of the antibody and/or enhance the delivery of an antibody across an epithelial barrier to the immune system. Examples of suitable effector molecules of this type include polymers, albumin, albumin binding proteins or albumin binding compounds such as those described in WO05/117984.

In one embodiment a half-life provided by an effector molecule which is independent of FcRn is advantageous.

Where the effector molecule is a polymer it may, in general, be a synthetic or a naturally occurring polymer, for example an optionally substituted straight or branched chain polyalkylene, polyalkenylene or polyoxyalkylene polymer or a branched or unbranched polysaccharide, e.g. a homo- or hetero- polysaccharide.

Specific optional substituents which may be present on the above-mentioned synthetic polymers include one or more hydroxy, methyl or methoxy groups.

Specific examples of synthetic polymers include optionally substituted straight or branched chain poly(ethyleneglycol), poly(propyleneglycol) poly(vinylalcohol) or derivatives thereof, especially optionally substituted poly(ethyleneglycol) such as methoxypoly(ethyleneglycol) or derivatives thereof.

Specific naturally occurring polymers include lactose, amylose, dextran, glycogen or derivatives thereof.

In one embodiment the polymer is albumin or a fragment thereof, such as human serum albumin or a fragment thereof.

“Derivatives” as used herein is intended to include reactive derivatives, for example thiol-selective reactive groups such as maleimides and the like. The reactive group may be linked directly or through a linker segment to the polymer. It will be appreciated that the residue of such a group will in some instances form part of the product as the linking group between the antibody fragment and the polymer.

The size of the polymer may be varied as desired, but will generally be in an average molecular weight range from 500 Da to 50000 Da, for example from 5000 to 40000 Da such as from 20000 to 40000 Da. The polymer size may in particular be selected on the basis of the intended use of the product for example ability to localize to certain tissues such as tumors or extend circulating half-life (for review see Chapman, 2002, Advanced Drug Delivery Reviews, 54, 531-545). Thus, for example, where the product is intended to leave the circulation and penetrate tissue, for example for use in the treatment of a tumour, it may be advantageous to use a small molecular weight polymer, for example with a molecular weight of around 5000 Da. For applications where the product remains in the circulation, it may be advantageous to use a higher molecular weight polymer, for example having a molecular weight in the range from 20000 Da to 40000 Da.

Suitable polymers include a polyalkylene polymer, such as a poly(ethyleneglycol) or, especially, a methoxypoly(ethyleneglycol) or a derivative thereof, and especially with a molecular weight in the range from about 15000 Da to about 40000 Da.

In one example antibodies for use in the present invention are attached to poly(ethyleneglycol) (PEG) moieties. In one particular example the antibody is an antibody fragment and the PEG molecules may be attached through any available amino acid side-chain or terminal amino acid functional group located in the antibody fragment, for example any free amino, imino, thiol, hydroxyl or carboxyl group. Such amino acids may occur naturally in the antibody fragment or may be engineered into the fragment using recombinant DNA methods (see for example U.S. Pat. No. 5,219,996; U.S. Pat. No. 5,667,425; WO98/25971, WO2008/038024). In one example the antibody molecule of the present invention is a modified Fab fragment wherein the modification is the addition to the C-terminal end of its heavy chain one or more amino acids to allow the attachment of an effector molecule. Suitably, the additional amino acids form a modified hinge region containing one or more cysteine residues to which the effector molecule may be attached. Multiple sites can be used to attach two or more PEG molecules.

Suitably PEG molecules are covalently linked through a thiol group of at least one cysteine residue located in the antibody fragment. Each polymer molecule attached to the modified antibody fragment may be covalently linked to the sulphur atom of a cysteine residue located in the fragment. The covalent linkage will generally be a disulphide bond or, in particular, a sulphur-carbon bond. Where a thiol group is used as the point of attachment appropriately activated effector molecules, for example thiol selective derivatives such as maleimides and cysteine derivatives may be used. An activated polymer may be used as the starting material in the preparation of polymer-modified antibody fragments as described above. The activated polymer may be any polymer containing a thiol reactive group such as an α-halocarboxylic acid or ester, e.g. iodoacetamide, an imide, e.g. maleimide, a vinyl sulphone or a disulphide. Such starting materials may be obtained commercially (for example from Nektar, formerly Shearwater Polymers Inc., Huntsville, Ala., USA) or may be prepared from commercially available starting materials using conventional chemical procedures. Particular PEG molecules include 20K methoxy-PEG-amine (obtainable from Nektar, formerly Shearwater; Rapp Polymere; and SunBio) and M-PEG-SPA (obtainable from Nektar, formerly Shearwater).

In one embodiment, the antibody is a modified Fab fragment, Fab′ fragment or diFab which is PEGylated, i.e. has PEG (poly(ethyleneglycol)) covalently attached thereto, e.g. according to the method disclosed in EP 0948544 or EP1090037 [see also “Poly(ethyleneglycol) Chemistry, Biotechnical and Biomedical Applications”, 1992, J. Milton Harris (ed), Plenum Press, New York, “Poly(ethyleneglycol) Chemistry and Biological Applications”, 1997, J. Milton Harris and S. Zalipsky (eds), American Chemical Society, Washington DC and “Bioconjugation Protein Coupling Techniques for the Biomedical Sciences”, 1998, M. Aslam and A. Dent, Grove Publishers, New York; Chapman, A. 2002, Advanced Drug Delivery Reviews 2002, 54:531-545]. In one example PEG is attached to a cysteine in the hinge region. In one example, a PEG modified Fab fragment has a maleimide group covalently linked to a single thiol group in a modified hinge region. A lysine residue may be covalently linked to the maleimide group and to each of the amine groups on the lysine residue may be attached a methoxypoly(ethyleneglycol) polymer having a molecular weight of approximately 20,000 Da. The total molecular weight of the PEG attached to the Fab fragment may therefore be approximately 40,000 Da.

Particular PEG molecules include 2-[3-(N-maleimido)propionamido]ethyl amide of N,N′-bis(methoxypoly(ethylene glycol) MW 20,000) modified lysine, also known as PEG2MAL4OK (obtainable from Nektar, formerly Shearwater).

Alternative sources of PEG linkers include NOF who supply GL2-400MA3 (wherein m in the structure below is 5) and GL2-400MA (where m is 2) and n is approximately 450:

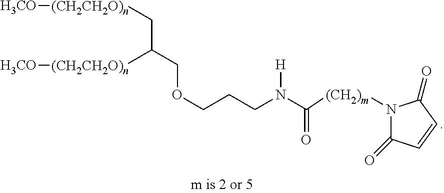

That is to say each PEG is about 20,000 Da.

Thus in one embodiment the PEG is 2,3-Bis(methylpolyoxyethylene-oxy)-1-{[3-(6-maleimido-1-oxohexyl)amino]propyloxy} hexane (the 2 arm branched PEG, —CH₂)₃NHCO(CH₂)₅-MAL, Mw 40,000 known as SUNBRIGHT GL2-400MA3.

Further alternative PEG effector molecules of the following type:

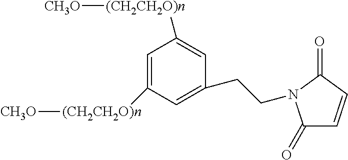

are available from Dr Reddy, NOF and Jenkem.

In one embodiment the antibody or fragment is conjugated to a starch molecule, for example to increase the half life. Methods of conjugating starch to a protein as described in U.S. Pat. No. 8,017,739 incorporated herein by reference.

In one embodiment there is provided an anti-FcRn binding molecule (i.e an antibody or binding fragment thereof) which:
Causes 50-85% reduction, such as a 70% reduction of plasma IgG concentration,
With not more than 25% or 20% reduction of plasma albumin concentration, and/or
With the possibility of repeat dosing to achieve long-term maintenance of low plasma IgG concentration.

The present invention also provides an isolated DNA sequence encoding the heavy and/or light chain(s) of an antibody molecule of the present invention. Suitably, the DNA sequence encodes the heavy or the light chain of an antibody molecule of the present invention. The DNA sequence of the present invention may comprise synthetic DNA, for instance produced by chemical processing, cDNA, genomic DNA or any combination thereof.

DNA sequences which encode an antibody molecule of the present invention can be obtained by methods well known to those skilled in the art. For example, DNA sequences coding for part or all of the antibody heavy and light chains may be synthesised as desired from the determined DNA sequences or on the basis of the corresponding amino acid sequences.

DNA coding for acceptor framework sequences is widely available to those skilled in the art and can be readily synthesised on the basis of their known amino acid sequences.

Standard techniques of molecular biology may be used to prepare DNA sequences coding for the antibody molecule of the present invention. Desired DNA sequences may be synthesised completely or in part using oligonucleotide synthesis techniques. Site-directed mutagenesis and polymerase chain reaction (PCR) techniques may be used as appropriate.

Examples of suitable DNA sequences are provided in herein.

Accordingly in one example the present invention provides an isolated DNA sequence encoding the heavy chain of an antibody Fab-dsscFv of the present invention which comprises the sequence given in SEQ ID NO: 13. Also provided is an isolated DNA sequence encoding the light chain of an antibody Fab-dsscFv of the present invention which comprises the sequence given in SEQ ID NO: 11.

The present invention also relates to a cloning or expression vector comprising one or more DNA sequences of the present invention. Accordingly, provided is a cloning or expression vector comprising one or more DNA sequences encoding an antibody of the present invention. Suitably, the cloning or expression vector comprises two DNA sequences, encoding the light chain and the heavy chain of the antibody molecule of the present invention, respectively and suitable signal sequences. In one example the vector comprises an intergenic sequence between the heavy and the light chains (see WO03/048208).

General methods by which the vectors may be constructed, transfection methods and culture methods are well known to those skilled in the art. In this respect, reference is made to “Current Protocols in Molecular Biology”, 1999, F. M. Ausubel (ed), Wiley Interscience, New York and the Maniatis Manual produced by Cold Spring Harbor Publishing.

Also provided is a host cell comprising one or more cloning or expression vectors comprising one or more DNA sequences encoding an antibody molecule of the present invention. Accordingly the present invention also provides a host cell for expression of an antibody molecule according to to the invention comprising:
i) a DNA sequence encoding the heavy chain of said antibody, and
ii) a DNA sequence encoding the light chain of said antibody

wherein the DNA sequences are provided in one or more cloning or expression vectors.

Any suitable host cell/vector system may be used for expression of the DNA sequences encoding the antibody molecule of the present invention. Bacterial, for example E. coli, and other microbial systems may be used (especially for expressing antibody fragments or eukaryotic, for example mammalian, host cell expression systems may also be used (especially for expressing full-length antibodies). Suitable mammalian host cells include CHO, myeloma or hybridoma cells.

Suitable types of Chinese Hamster Ovary (CHO cells) for use in the present invention may include CHO and CHO-K1 cells including dhfr-CHO cells, such as CHO-DG44 cells and CHO-DXB11 cells, which may be used with a DHFR selectable marker or CHOK1-SV cells which may be used with a glutamine synthetase selectable marker. Other cell types of use in expressing antibodies include lymphocytic cell lines, e.g., NSO myeloma cells and SP2 cells, COS cells.

The present invention also provides a process for the production of an antibody molecule according to the present invention comprising culturing a host cell containing a vector or vectors of the present invention under conditions suitable for leading to expression of protein from DNA encoding the antibody molecule of the present invention, and isolating the antibody molecule.

The antibody molecule comprises both heavy and light chains and the cell line may be transfected with two vectors, a first vector encoding a light chain polypeptide and a second vector encoding a heavy chain polypeptide. Alternatively, a single vector may be used, the vector including sequences encoding light chain and heavy chain polypeptides.

The antibodies and fragments according to the present disclosure are expressed at good levels from host cells. Thus the properties of the antibodies and/or fragments are conducive to commercial processing.

Thus there is a provided a process for culturing a host cell and expressing an antibody or fragment thereof, isolating the latter and optionally purifying the same to provide an isolated antibody or fragment. In one embodiment the process further comprises the step of conjugating an effector molecule to the isolated antibody or fragment, for example conjugating to a PEG polymer in particular as described herein.

In one embodiment there is provided a process for purifiying an antibody (in particular an antibody or fragment according to the invention) comprising the steps: performing anion exchange chromatography in non-binding mode such that the impurities are retained on the column and the antibody is eluted.

In one embodiment the purification employs affinity capture on an FcRn column.

In one embodiment the purification employs cibacron blue or similar for purification of albumin fusion or conjugate molecules.

Suitable ion exchange resins for use in the process include Q.FF resin (supplied by GE-Healthcare). The step may, for example be performed at a pH about 8.

The process may further comprise an initial capture step employing cation exchange chromatography, performed for example at a pH of about 4 to 5, such as 4.5. The cation exchange chromatography may, for example employ a resin such as CaptoS resin or SP sepharose FF (supplied by GE-Healthcare). The antibody or fragment can then be eluted from the resin employing an ionic salt solution such as sodium chloride, for example at a concentration of 200 mM.

Thus the chromatograph step or steps may include one or more washing steps, as appropriate.

The purification process may also comprise one or more filtration steps, such as a diafiltration step.

Thus in one embodiment there is provided a purified anti-FcRn antibody or fragment, for example a humanised antibody or fragment, in particular an antibody or fragment according to the invention, in substantially purified from, in particular free or substantially free of endotoxin and/or host cell protein or DNA.

Purified form as used supra is intended to refer to at least 90% purity, such as 91, 92, 93, 94, 95, 96, 97, 98, 99% w/w or more pure.

Substantially free of endotoxin is generally intended to refer to an endotoxin content of 1 EU per mg antibody product or less such as 0.5 or 0.1 EU per mg product.

Substantially free of host cell protein or DNA is generally intended to refer to host cell protein and/or DNA content 400 μg per mg of antibody product or less such as 100 μg per mg or less, in particular 20 μg per mg, as appropriate.

The antibody molecules of the present invention may also be used in diagnosis, for example in the in vivo diagnosis and imaging of disease states involving FcRn.

As the antibodies of the present invention are useful in the treatment and/or prophylaxis of a pathological condition, the present invention also provides a pharmaceutical or diagnostic composition comprising an antibody molecule of the present invention in combination with one or more of a pharmaceutically acceptable excipient, diluent or carrier. Accordingly, provided is the use of an antibody molecule of the invention for the manufacture of a medicament. The composition will usually be supplied as part of a sterile, pharmaceutical composition that will normally include a pharmaceutically acceptable carrier. A pharmaceutical composition of the present invention may additionally comprise a pharmaceutically-acceptable excipient.

The present invention also provides a process for preparation of a pharmaceutical or diagnostic composition comprising adding and mixing the antibody molecule of the present invention together with one or more of a pharmaceutically acceptable excipient, diluent or carrier.

The antibody molecule may be the sole active ingredient in the pharmaceutical or diagnostic composition or may be accompanied by other active ingredients including other antibody ingredients or non-antibody ingredients such as steroids or other drug molecules, in particular drug molecules whose half-life is independent of FcRn binding.

The pharmaceutical compositions suitably comprise a therapeutically effective amount of the antibody of the invention. The term “therapeutically effective amount” as used herein refers to an amount of a therapeutic agent needed to treat, ameliorate or prevent a targeted disease or condition, or to exhibit a detectable therapeutic or preventative effect. For any antibody molecule, the therapeutically effective amount can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans.

The precise therapeutically effective amount for a human subject will depend upon the severity of the disease state, the general health of the subject, the age, weight and gender of the subject, diet, time and frequency of administration, drug combination(s), reaction sensitivities and tolerance/response to therapy. This amount can be determined by routine experimentation and is within the judgement of the clinician. Generally, a therapeutically effective amount will be from 0.01 mg/kg to 500 mg/kg, for example 0.1 mg/kg to 200 mg/kg, such as 100mg/Kg.

Pharmaceutical compositions may be conveniently presented in unit dose forms containing a predetermined amount of an active agent of the invention per dose.

Therapeutic doses of the antibodies according to the present disclosure show no apparent toxicology effects in vivo.

In one embodiment of an antibody or fragment according to the invention a single dose may provide up to a 70% reduction in circulating IgG levels. In one example of an antibody or fragment according to the invention a single dose may provide up to a 80% reduction in circulating IgG levels. In one example of an antibody or fragment according to the invention a single dose may provide a greater than 80% reduction in circulating IgG levels.

The maximal therapeutic reduction in circulating IgG may be observed about 1 week after administration of the relevant therapeutic dose. The levels of IgG may recover over the weeks following dosing if further therapeutic doses are not delivered. Recover as employed herein refers to levels returning to levels similar to those observed before initial dosing commenced.

Advantageously, the levels of IgG in vivo may be maintained at an appropriately low level by administration of sequential doses of the antibody or fragments according to the disclosure.

Compositions may be administered individually to a patient or may be administered in combination (e.g. simultaneously, sequentially or separately) with other agents, drugs or hormones.

Agents as employed herein refers to an entity which when administered has a physiological affect.

Drug as employed herein refers to a chemical entity which at a therapeutic dose has an appropriate physiological affect.

In one embodiment the antibodies or fragments according to the present disclosure are employed with an immunosuppressant therapy, such as a steroid, in particular prednisone.

In one embodiment the antibodies or fragments according to the present disclosure are employed with Rituximab or other B cell therapies.

In one embodiment the antibodies or fragments according to the present disclosure are employed with any B cell or T cell modulating agent or immunomodulator. Examples include methotrexate, microphenyolate and azathioprine.

The dose at which the antibody molecule of the present invention is administered depends on the nature of the condition to be treated, the extent of the inflammation present and on whether the antibody molecule is being used prophylactically or to treat an existing condition.

The frequency of dosing will depend on the half life of the antibody, its target-mediated disposition, the duration of its effect, and the presence of anti-drug antibodies. If the antibody has a short half life (a few hours) or a limited activity, and/or if it is desirable to deliver small volumes of drug (e.g. for subcutaneous injection), it may be necessary to dose frequently, as frequently as once or more per day. Alternatively, if the antibody has a long half life, has long duration of activity, or can be dosed in large volumes (such as by infusion) dosing may be infrequent, once per day, or every few days, weeks or months. In one embodiment, sufficient time is allowed between doses to allow anti-drug antibody levels to decline.

Half life as employed herein is intended to refer to the duration of the molecule in circulation, for example in serum/plasma.

Pharmacodynamics as employed herein refers to the profile and in particular duration of the biological action of the molecule according the present disclosure.

The pharmaceutically acceptable carrier should not itself induce the production of antibodies harmful to the individual receiving the composition and should not be toxic. Suitable carriers may be large, slowly metabolised macromolecules such as proteins, polypeptides, liposomes, polysaccharides, polylactic acids, polyglycolic acids, polymeric amino acids, amino acid copolymers and inactive virus particles.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in therapeutic compositions may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the patient.

Suitable forms for administration include forms suitable for parenteral administration, e.g. by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilising and/or dispersing agents. Alternatively, the antibody molecule may be in dry form, for reconstitution before use with an appropriate sterile liquid.

Once formulated, the compositions of the invention can be administered directly to the subject. The subjects to be treated can be animals. However, in one or more embodiments the compositions are adapted for administration to human subjects.

Suitably in formulations according to the present disclosure, the pH of the final formulation is not similar to the value of the isoelectric point of the antibody or fragment, for example if the pI of the protein is in the range 8-9 or above then a formulation pH of 7 may be appropriate. Whilst not wishing to be bound by theory it is thought that this may ultimately provide a final formulation with improved stability, for example the antibody or fragment remains in solution.

In one example the pharmaceutical formulation at a pH in the range of 4.0 to 7.0 comprises: 1 to 200 mg/mL of an antibody molecule according to the present disclosure, 1 to 100 mM of a buffer, 0.001 to 1% of a surfactant, a) 10 to 500 mM of a stabiliser, b) 10 to 500 mM of a stabiliser and 5 to 500 mM of a tonicity agent, or c) 5 to 500 mM of a tonicity agent.

The pharmaceutical compositions of this invention may be administered by any number of routes including, but not limited to, oral, intravenous, intramuscular, intra-arterial, intramedullary, intrathecal, intraventricular, transdermal, transcutaneous (for example, see WO98/20734), subcutaneous, intraperitoneal, intranasal, enteral, topical, sublingual, intravaginal or rectal routes. Hyposprays may also be used to administer the pharmaceutical compositions of the invention. Typically, the therapeutic compositions may be prepared as injectables, either as liquid solutions or suspensions. Solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule.

It will be appreciated that the active ingredient in the composition will be an antibody molecule. As such, it will be susceptible to degradation in the gastrointestinal tract. Thus, if the composition is to be administered by a route using the gastrointestinal tract, the composition will need to contain agents which protect the antibody from degradation but which release the antibody once it has been absorbed from the gastrointestinal tract.

A thorough discussion of pharmaceutically acceptable carriers is available in Remington's Pharmaceutical Sciences (Mack Publishing Company, N.J. 1991).

In one embodiment the formulation is provided as a formulation for topical administrations including inhalation.

Suitable inhalable preparations include inhalable powders, metering aerosols containing propellant gases or inhalable solutions free from propellant gases. Inhalable powders according to the disclosure containing the active substance may consist solely of the abovementioned active substances or of a mixture of the abovementioned active substances with physiologically acceptable excipient.

These inhalable powders may include monosaccharides (e.g. glucose or arabinose), disaccharides (e.g. lactose, saccharose, maltose), oligo- and polysaccharides (e.g. dextranes), polyalcohols (e.g. sorbitol, mannitol, xylitol), salts (e.g. sodium chloride, calcium carbonate) or mixtures of these with one another. Mono- or disaccharides are suitably used, the use of lactose or glucose, particularly but not exclusively in the form of their hydrates.

Particles for deposition in the lung require a particle size less than 10 microns, such as 1-9 microns for example from 1 to 5 μm. The particle size of the active ingredient (such as the antibody or fragment) is of primary importance.

The propellent gases which can be used to prepare the inhalable aerosols are known in the art. Suitable propellent gases are selected from among hydrocarbons such as n-propane, n-butane or isobutane and halohydrocarbons such as chlorinated and/or fluorinated derivatives of methane, ethane, propane, butane, cyclopropane or cyclobutane. The abovementioned propellent gases may be used on their own or in mixtures thereof.

Particularly suitable propellent gases are halogenated alkane derivatives selected from among TG 11, TG 12, TG 134a and TG227. Of the abovementioned halogenated hydrocarbons, TG134a (1,1,1,2-tetrafluoroethane) and TG227 (1,1,1,2,3,3,3-heptafluoropropane) and mixtures thereof are particularly suitable.

The propellent-gas-containing inhalable aerosols may also contain other ingredients such as cosolvents, stabilisers, surface-active agents (surfactants), antioxidants, lubricants and means for adjusting the pH. All these ingredients are known in the art.

The propellant-gas-containing inhalable aerosols according to the invention may contain up to 5% by weight of active substance. Aerosols according to the invention contain, for example, 0.002 to 5% by weight, 0.01 to 3% by weight, 0.015 to 2% by weight, 0.1 to 2% by weight, 0.5 to 2% by weight or 0.5 to 1% by weight of active ingredient.

Alternatively topical administrations to the lung may also be by administration of a liquid solution or suspension formulation, for example employing a device such as a nebulizer, for example, a nebulizer connected to a compressor (e.g., the Pari LC-Jet Plus(R) nebulizer connected to a Pari Master(R) compressor manufactured by Pari Respiratory Equipment, Inc., Richmond, Va.).

The antibody of the invention can be delivered dispersed in a solvent, e.g., in the form of a solution or a suspension. It can be suspended in an appropriate physiological solution, e.g., saline or other pharmacologically acceptable solvent or a buffered solution. Examples of buffered solutions known in the art may contain 0.05 mg to 0.15 mg disodium edetate, 8.0 mg to 9.0 mg NaCl, 0.15 mg to 0.25 mg polysorbate, 0.25 mg to 0.30 mg anhydrous citric acid, and 0.45 mg to 0.55 mg sodium citrate per 1 ml of water so as to achieve a pH of about 4.0 to 5.0. A suspension can employ, for example, lyophilised antibody.

The therapeutic suspensions or solution formulations can also contain one or more excipients. Excipients are well known in the art and include buffers (e.g., citrate buffer, phosphate buffer, acetate buffer and bicarbonate buffer), amino acids, urea, alcohols, ascorbic acid, phospholipids, proteins (e.g., serum albumin), EDTA, sodium chloride, liposomes, mannitol, sorbitol, and glycerol. Solutions or suspensions can be encapsulated in liposomes or biodegradable microspheres. The formulation will generally be provided in a substantially sterile form employing sterile manufacture processes.

This may include production and sterilization by filtration of the buffered solvent/solution used for the formulation, aseptic suspension of the antibody in the sterile buffered solvent solution, and dispensing of the formulation into sterile receptacles by methods familiar to those of ordinary skill in the art.

Nebulizable formulation according to the present disclosure may be provided, for example, as single dose units (e.g., sealed plastic containers or vials) packed in foil envelopes. Each vial contains a unit dose in a volume, e.g., 2 mL, of solvent/solution buffer.

The antibodies disclosed herein may be suitable for delivery via nebulisation.

It is also envisaged that the antibody of the present invention may be administered by use of gene therapy. In order to achieve this, DNA sequences encoding the heavy and light chains of the antibody molecule under the control of appropriate DNA components are introduced into a patient such that the antibody chains are expressed from the DNA sequences and assembled in situ.

The present invention also provides an antibody molecule (or compositions comprising same) for use in the control of autoimmune diseases, for example Acute Disseminated Encephalomyelitis (ADEM), Acute necrotizing hemorrhagic leukoencephalitis, Addison's disease, Agammaglobulinemia, Alopecia areata, Amyloidosis, ANCA-associated vasculitis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune pancreatitis, Autoimmune retinopathy, Autoimmune thrombocytopenic purpura (ATP), Autoimmune thyroid disease, Autoimmune urticarial, Axonal & nal neuropathies, Balo disease, Behcet's disease, Bullous pemphigoid, Cardiomyopathy, Castleman disease, Celiac disease, Chagas disease, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Dilated cardiomyopathy, Discoid lupus, Dressler's syndrome, Endometriosis, Eosinophilic angiocentric fibrosis, Eosinophilic fasciitis, Erythema nodosum, Experimental allergic encephalomyelitis, Evans syndrome, Fibrosing alveolitis, Giant cell arteritis (temporal arteritis), Glomerulonephritis, Goodpasture's syndrome, Granulomatosis with Polyangiitis (GPA) see Wegener's, Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic hypocomplementemic tubulointestitial nephritis, Idiopathic thrombocytopenic purpura (ITP), IgA nephropathy, IgG4-related disease, IgG4-related sclerosing disease, Immunoregulatory lipoproteins, Inflammatory aortic aneurysm, Inflammatory pseudotumour, Inclusion body myositis, Insulin-dependent diabetes (type 1), Interstitial cystitis, Juvenile arthritis, Juvenile diabetes, Kawasaki syndrome, Kuttner's tumour, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Mediastinal fibrosis, Meniere's disease, Microscopic polyangiitis, Mikulicz's syndrome, Mixed connective tissue disease (MCTD), Mooren's ulcer, Mucha-Habermann disease, Multifocal fibrosclerosis, Multiple sclerosis, Myasthenia gravis, Myositis, Narcolepsy, Neuromyelitis optica (Devic's), Neutropenia, Ocular cicatricial pemphigoid, Optic neuritis, Ormond's disease (retroperitoneal fibrosis), Palindromic rheumatism, PANDAS (Pediatric Autoimmune Neuropsychiatric Disorders Associated with Streptococcus), Paraneoplastic cerebellar degeneration, Paraproteinemic polyneuropathies, Paroxysmal nocturnal hemoglobinuria (PNH), Parry Romberg syndrome, Parsonnage-Turner syndrome, Pars planitis (peripheral uveitis), Pemphigus vulgaris, Periaortitis, Periarteritis, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatic, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reflex sympathetic dystrophy, Reiter's syndrome, Relapsing polychondritis, Restless legs syndrome, Retroperitoneal fibrosis (Ormond's disease), Rheumatic fever, Rheumatoid arthritis, Riedel's thyroiditis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombotic, thrombocytopenic purpura (TTP), Tolosa-Hunt syndrome, Transverse myelitis, Ulcerative colitis, Undifferentiated connective tissue disease (UCTD), Uveitis, Vasculitis, Vesiculobullous dermatosis, Vitiligo, Waldenstrom Macroglobulinaemia, Warm idiopathic haemolytic anaemia and Wegener's granulomatosis (now termed Granulomatosis with Polyangiitis (GPA).

Additional indications may also include hyperviscosity syndromes; cryoglobulinemia; recurrent focal and segmental glomerulosclerosis in the transplanted kidney; HELLP syndrome; Refsum disease; HIV-related neuropathy; rhabdomyolysis and alloimune diseases.

In one embodiment the antibodies or fragments according to the disclosure are employed in the treatment or prophylaxis of epilepsy or seizures.

In one embodiment the antibodies or fragments according to the disclosure are employed in the treatment or prophylaxis of multiple sclerosis.

In embodiment the antibodies and fragments of the disclosure are employed in alloimmune disease/indications which includes:
Transplantation donor mismatch due to anti-HLA antibodies
Foetal and neonatal alloimmune thrombocytopenia, FNAIT (or neonatal alloimmune thrombocytopenia, NAITP or NAIT or NAT, or foeto-maternal alloimmune thrombocytopenia, FMAITP or FMAIT).

Additional indications include: rapid clearance of Fc-containing biopharmaceutical drugs from human patients and combination of anti-FcRn therapy with other therapies—IVIg, Rituxan, plasmapheresis. For example anti-FcRn therapy may be employed following Rituxan therapy. In addition anti-FcRn therapy may be used to rapidly clear imaging agents such as radiolabelled antibodies used in imaging tumors.

In one embodiment the antibodies and fragments of the disclosure are employed in a neurology disorder such as:
Chronic inflammatory demyelinating polyneuropathy (CIDP)
Guillain-Barre syndrome
Paraproteinemic polyneuropathies
Neuromyelitis optica (NMO, NMO spectrum disorders or NMO spectrum diseases), and
Myasthenia gravis.

In one embodiment the antibodies and fragments of the disclosure are employed in a dermatology disorder such as:
Bullous pemphigoid
Pemphigus vulgaris
ANCA-associated vasculitis
Dilated cardiomyopathy

In one embodiment the antibodies and fragments of the disclosure are employed in an Immunology, haematology disorder such as:
Idiopathic thrombocytopenic purpura (ITP)
Thrombotic thrombocytopenic purpura (TTP)
Warm idiopathic haemolytic anaemia
Goodpasture's syndrome
Transplantation donor mismatch due to anti-HLA antibodies

In one embodiment the disorder is selected from Myasthenia Gravis, Neuro- myelitis Optica, CIDP, Guillaume-Barre Syndrome, Para-proteinemic Poly neuropathy, Refractory Epilepsy, ITP/TTP, Hemolytic Anemia, Goodpasture's Syndrome, ABO mismatch, Lupus nephritis, Renal Vasculitis, Sclero-derma, Fibrosing alveolitis, Dilated cardio-myopathy, Grave's Disease, Type 1 diabetes, Auto-immune diabetes, Pemphigus, Sclero-derma, Lupus, ANCA vasculitis, Dermato-myositis, Sjogren's Disease and Rheumatoid Arthritis.

In one embodiment the disorder is selected from autoimmune polyendocrine syndrome types 1 (APECED or Whitaker's Syndrome) and 2 (Schmidt's Syndrome); alopecia universalis; myasthenic crisis; thyroid crisis; thyroid associated eye disease; thyroid ophthalmopathy; autoimmune diabetes; autoantibody associated encephalitis and/or encephalopathy; pemphigus foliaceus; epidermolysis bullosa; dermatitis herpetiformis; Sydenham's chorea; acute motor axonal neuropathy (AMAN); Miller-Fisher syndrome; multifocal motor neuropathy (MMN); opsoclonus; inflammatory myopathy; Isaac's syndrome (autoimmune neuromyotonia), Paraneoplastic syndromes and Limbic encephalitis.

The antibodies and fragments according to the present disclosure may be employed in treatment or prophylaxis.

The present invention also provides a method of reducing the concentration of undesired antibodies in an individual comprising the steps of administering to an individual a therapeutically effective dose of an anti-FcRn antibody fusion protein described herein. The present invention further provides the use of an antibody molecule according to the present invention in the manufacture of a medicament for the treatment and/or prophylaxis of a pathological disorder described herein such as an autoimmune disease.

Accordingly the present invention also provides an antibody molecule (or compositions comprising same) for use in the control of an autoimmune disease selected from the group consisting of Acute Disseminated Encephalomyelitis (ADEM), Acute necrotizing hemorrhagic leukoencephalitis, Addison's disease, Agammaglobulinemia, Alopecia areata, Amyloidosis, ANCA-associated vasculitis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency , Autoimmune inner ear disease (AIED), Autoimmune myocarditis, Autoimmune pancreatitis, Autoimmune retinopathy, Autoimmune thrombocytopenic purpura (ATP), Autoimmune thyroid disease, Autoimmune urticarial, Axonal & nal neuropathies, Balo disease, Behcet's disease, Bullous pemphigoid, Cardiomyopathy, Castleman disease, Celiac disease, Chagas disease, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Dilated cardiomyopathy, Discoid lupus, Dressler's syndrome, Endometriosis, Eosinophilic angiocentric fibrosis, Eosinophilic fasciitis, Erythema nodosum, Experimental allergic encephalomyelitis, Evans syndrome, Fibrosing alveolitis, Giant cell arteritis (temporal arteritis), Glomerulonephritis, Goodpasture's syndrome, Granulomatosis with Polyangiitis (GPA) see Wegener's, Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic hypocomplementemic tubulointestitial nephritis, Idiopathic thrombocytopenic purpura (ITP), IgA nephropathy, IgG4-related disease, IgG4-related sclerosing disease, Immunoregulatory lipoproteins, Inflammatory aortic aneurysm, Inflammatory pseudotumour, Inclusion body myositis, Insulin-dependent diabetes (type 1), Interstitial cystitis, Juvenile arthritis, Juvenile diabetes, Kawasaki syndrome, Kuttner's tumour, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Mediastinal fibrosis, Meniere's disease, Microscopic polyangiitis, Mikulicz's syndrome, Mixed connective tissue disease (MCTD), Mooren's ulcer, Mucha-Habermann disease, Multifocal fibrosclerosis, Multiple sclerosis, Myasthenia gravis, Myositis, Narcolepsy, Neuromyelitis optica (Devic's), Neutropenia, Ocular cicatricial pemphigoid, Optic neuritis, Ormond's disease (retroperitoneal fibrosis), Palindromic rheumatism, PANDAS (Pediatric Autoimmune Neuropsychiatric Disorders Associated with Streptococcus), Paraneoplastic cerebellar degeneration, Paraproteinemic polyneuropathies, Paroxysmal nocturnal hemoglobinuria (PNH), Parry Romberg syndrome, Parsonnage-Turner syndrome, Pars planitis (peripheral uveitis), Pemphigus vulgaris, Periaortitis, Periarteritis, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatic, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reflex sympathetic dystrophy, Reiter's syndrome, Relapsing polychondritis, Restless legs syndrome, Retroperitoneal fibrosis (Ormond's disease), Rheumatic fever, Rheumatoid arthritis, Riedel's thyroiditis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombotic, thrombocytopenic purpura (TTP), Tolosa-Hunt syndrome, Transverse myelitis, Ulcerative colitis, Undifferentiated connective tissue disease (UCTD), Uveitis, Vasculitis, Vesiculobullous dermatosis, Vitiligo, Waldenstrom Macroglobulinaemia, Warm idiopathic haemolytic anaemia and Wegener's granulomatosis (now termed Granulomatosis with Polyangiitis (GPA).

In one embodiment the present disclosure comprises use of antibodies or fragments thereof as a reagent for diagnosis, for example conjugated to a reporter molecule. Thus there is provided antibody or fragment according to the disclosure which is labelled. In one aspect there is provided a column comprising an antibody or fragment according to the disclosure.

Thus there is provided an anti-FcRn antibody or binding fragment for use as a reagent for such uses as:
1) purification of FcRn protein (or fragments thereof)—being conjugated to a matrix and used as an affinity column, or (as a modified form of anti-FcRn) as a precipitating agent (e.g. as a form modified with a domain recognised by another molecule, which may be modified by addition of an Fc (or produced as full length IgG), which is optionally precipitated by an anti-Fc reagent)
2) detection and/or quantification of FcRn on cells or in cells, live or fixed (cells in vitro or in vivo in tissue or cell sections). Uses for this may include quantification of FcRn as a biomarker, to follow the effect of anti-FcRn treatment. For these purposes, the candidate might be used in a modified form (e.g. by addition of an Fc domain, as in full length IgG, or some other moiety, as a genetic fusion protein or chemical conjugate, such as addition of a fluorescent tag used for the purposes of detection).
3) purification or sorting of FcRn-bearing cells labeled by binding to candidate modified by ways exemplified in (1) and (2).

Also provided by the present invention is provided an assay suitable for assessing the ability of a test molecule such as an antibody molecule to block FcRn activity and in particular the ability of the cells to recycle IgG. Such an assay may be useful for identifying inhibitors of FcRn activity, such as antibody molecules or small molecules and as such may also be useful as a batch release assay in the production of such an inhibitor. The assay was previously described in WO2014/019727.

Comprising in the context of the present specification is intended to meaning including.

Where technically appropriate embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

The present invention is further described by way of illustration only in the following examples, which refer to the accompanying Figures, in which:

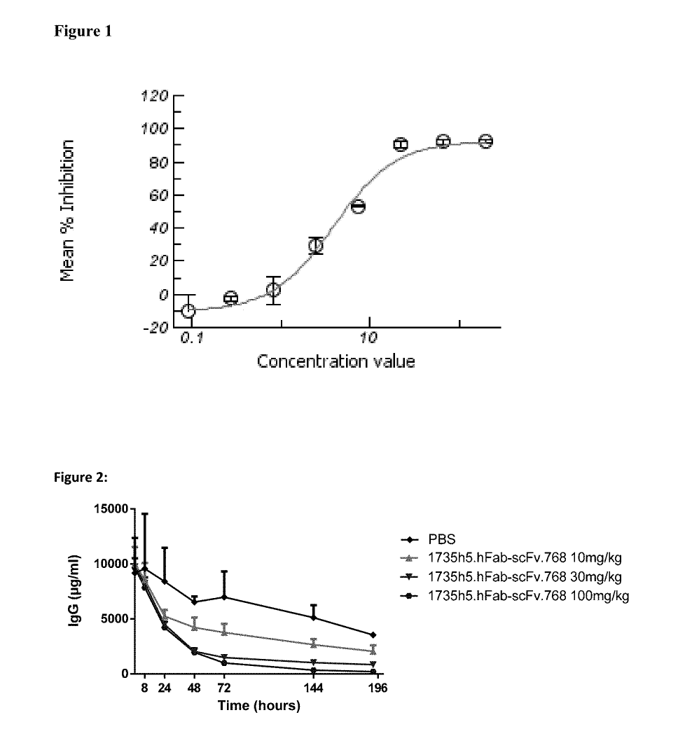

FIG. 1 shows 1735h5.hFab-scFv.768 inhibits IgG recycling in MDCK II clone 7 cells

FIG. 2 shows the effect of 1735h5.hFab-scFv.768 on the concentration of human IVIg in serum of human FcRn-trangenic mice

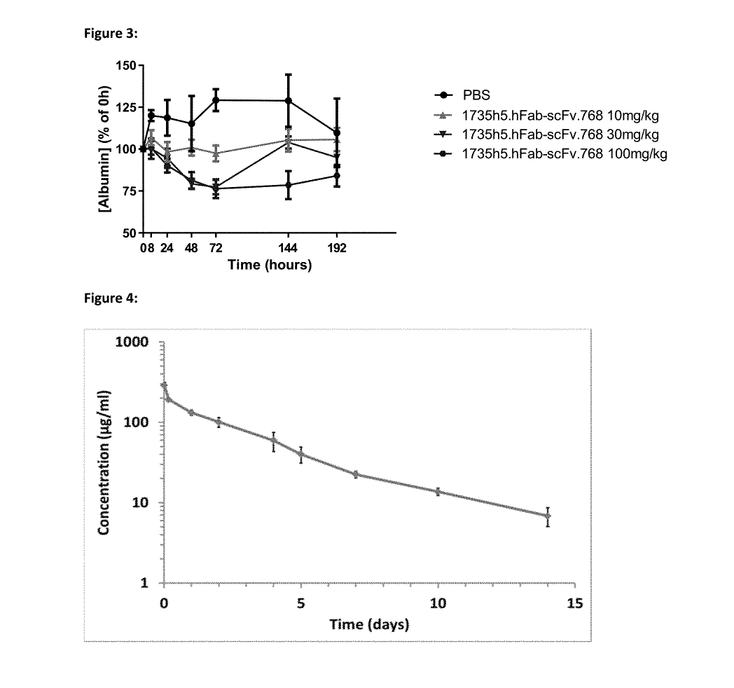

FIG. 3 shows the effect of 1735h5.hFab-scFv.768on the concentration of serum albumin in human FcRn-transgenic mice.

FIG. 4 shows the pharmacokinetics of 1735h5.hFab-scFv.768 in normal mice.

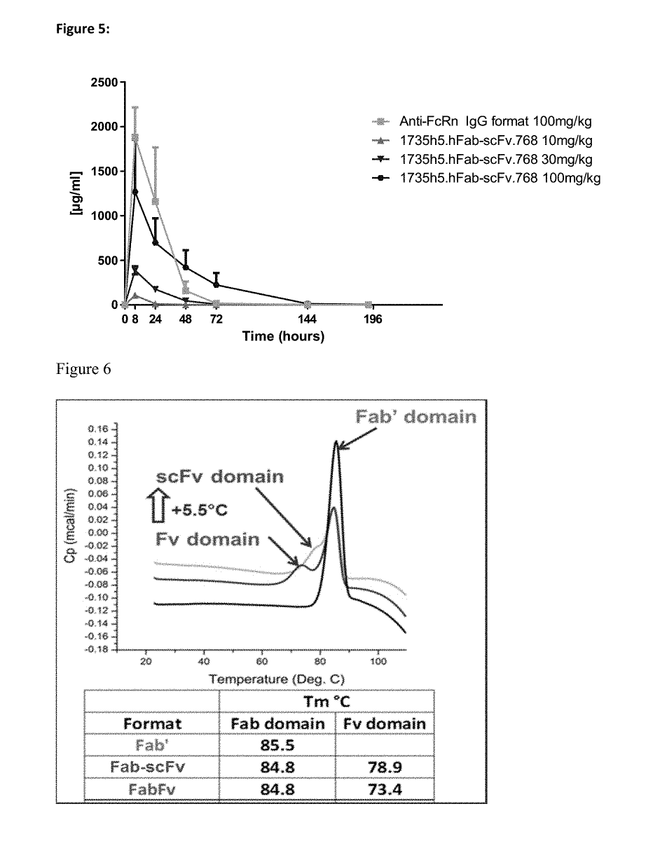

FIG. 5 shows the pharmacokinetics of 1735h5.hFab-scFv.768 in human FcRn-transgenic mice.

FIG. 6 shows thermal Stability of 1735h5.hFab-scFv.768 (Fab-scFv) compared with parent Fab and equivalent Fab-Fv .

FIG. 7 shows Fab-scFv and Fab-dsscFv fragment formats of the present disclosure

FIG. 8 Antibody sequences according to the present disclosure

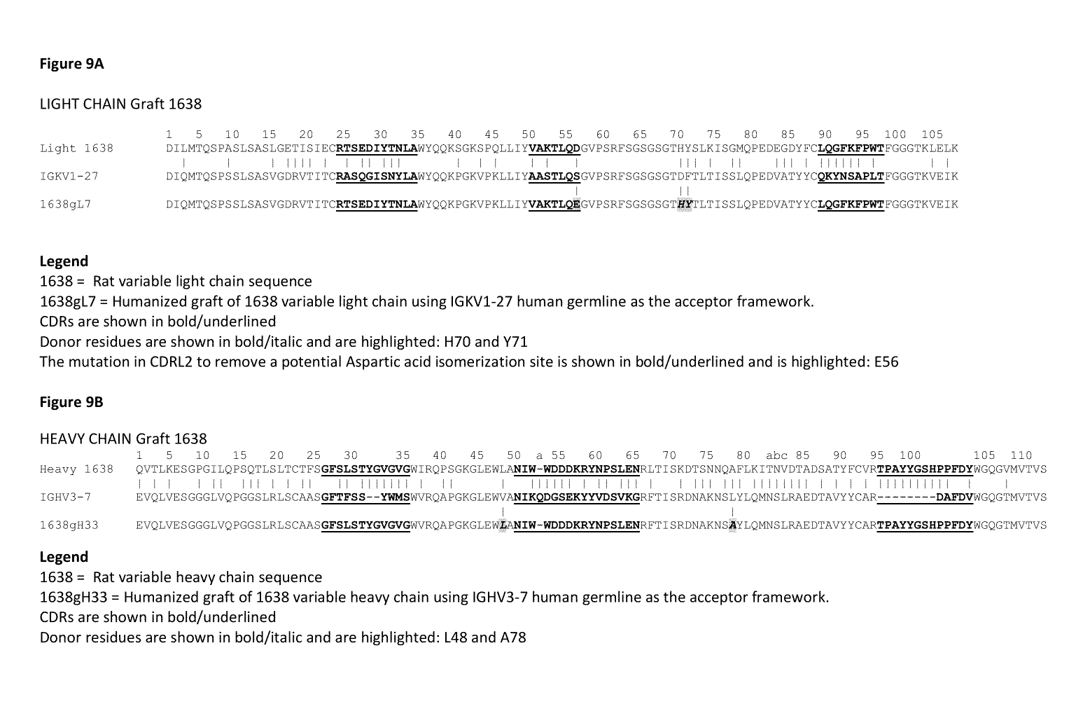

FIG. 9a Humanisation of antibody 1638.g49

FIG. 9b Humanisation of antibody 1638.g49

### EXAMPLES

### Abbreviations

° C. temperature, degrees centigrade.

### ATR FTIR Attenuated Total Reflectance Fourier Transform Infra-Red Spectroscopy

CH2 constant heavy chain region 2
cIEF capillary isoelectric focusing
DSC differential scanning calorimetry
GOF fucosylated aglactosyl biantennary glycan
H chain Heavy chain
HPLC high performance liquid chromatography
IgG immunoglobulin G
L chain Light chain
nLCMS nano-liquid chromatography mass spectrometry
PBS phosphate-buffered saline buffer
PI isoelectric point
SD standard deviation
SEC size exclusion chromatography
ToF time of flight
Tₘ melting temperature
TCEP tris(2-carboxyethyl)phosphine

### THP Tris(hydroxypropyl)phosphine

Tris tris(hydroxymethyl)aminomethane
The following immunizations were performed in order to generate material for B cell culture and antibody screening:
Sprague Dawley rats were immunized with three shots of NIH3T3 mouse fibroblasts co-expressing mutant human FcRn (L320A; L321A) (Ober et al., 2001 Int. Immunol. 13, 1551-1559) and mouse β2M with a fourth final boost of human FcRn extracellular domain. Sera were monitored for both binding to mutant FcRn on HEK-293 cells and for its ability to prevent binding of Alexafluor 488-labelled human IgG. Both methods were performed by flow cytometry. For binding, phycoerythrin (PE)-labelled anti mouse or rat Fc specific secondary reagents were used to reveal binding of IgG in sera.
B cell cultures were prepared using a method similar to that described by Zubler et al. (1985). Briefly, B cells at a density of approximately 5000 cells per well were cultured in bar-coded 96-well tissue culture plates with 200 μl/well RPMI 1640 medium (Gibco BRL) supplemented with 10% FCS (PAA laboratories ltd), 2% HEPES (Sigma Aldrich), 1% L-Glutamine (Gibco BRL), 1% penicillin/streptomycin solution (Gibco BRL), 0.1% β-mercaptoethanol (Gibco BRL), 2-5% activated rabbit splenocyte culture supernatant and gamma-irradiated EL-4-B5 murine thymoma cells (5×10⁴/well) for seven days at 37° C. in an atmosphere of 5% CO₂.
The presence of FcRn-specific antibodies in B cell culture supernatants was determined using a homogeneous fluorescence-based binding assay using HEK-293 cells transiently transfected with mutant FcRn (surface-stabilised) as a source of target antigen. 10 μl of supernatant was transferred from barcoded 96-well tissue culture plates into barcoded 384-well black-walled assay plates containing 5000 transfected HEK-293 cells per well using a Matrix Platemate liquid handler. Binding was revealed with a goat anti-rat or mouse IgG Fcy-specific Cy-5 conjugate (Jackson). Plates were read on an Applied Biosystems 8200 cellular detection system. From 3800×96-well culture plates, representing 38 different immunized animals, 9800 anti-human FcRn binders were identified. It was estimated that this represented the screening of approximately 2.5 billion B cells.
Following primary screening, positive supernatants were consolidated on 96-well bar-coded master plates using an Aviso Onyx hit-picking robot and B cells in cell culture plates frozen at −80C. Master plates were then screened in a Biacore assay in order to identify wells containing antibodies of high affinity and those which inhibited the binding of human IgG to FcRn (see below).
Biomolecular interaction analysis using surface plasmon resonance technology (SPR) was performed on a BIAcore T200 system (GE Healthcare). Goat anti-rat IgG, Fc gamma (Chemicon International Inc.) in 10 mM NaAc, pH 5 buffer was immobilized on a CMS Sensor Chip via amine coupling chemistry to a capture level of approx. 19500 response units (RU) using HBS-EP⁺ as the running buffer. 50 mM Phosphate, pH6+150 mM NaCl was used as the running buffer for the affinity and blocking assay. B cell culture supernatants were diluted 1 in 5 in 200 mM Phosphate, pH6+150 mM NaCl. A 600 s injection of diluted B cell supernatant at 5 μl/min was used for capture by the immobilized anti-rat IgG,Fc. Human FcRn at 100 nM was injected over the captured B cell culture supernatant for 180 s at 30 μl/min followed by 360 s dissociation. Human IgG (Jackson ImmunoResearch) was injected over for 60 s with 180 s dissociation at 30 μl/min.
The data was analysed using T200 evaluation software (version 1.0) to determine affinity constants (K_{D}) of antibodies and determine those which blocked IgG binding.
As an alternative assay, master plate supernatants were also screened in a cell-based human IgG blocking assay. 25 ul of B cell culture supernatant from master plates were added to 96 well U-bottomed polypropylene plate. Mutant hFcRn-transfected HEK-293 cells (50,000 cells per well in 25 ul PBS pH6/1% FCS) were then added to each well and incubated for 1 hour at 4° C. Cells were washed twice with 150 ul of PBS media. Cells were then resuspended in 50 ul/well PBS/FCS media containing human IgG labelled with Alexafluor 488 or 649 at 7.5 ug/ml and incubated 1 hour at 4° C. Cells were then washed twice with 150 ul of media and then resuspended in 35 ul/well of PBS/FCS media containing 1% formaldehyde as fixative. Plates were then read on a FACS Canto 2 flow cytometer.
To allow recovery of antibody variable region genes from a selection of wells of interest, a deconvolution step had to be performed to enable identification of the antigen-specific B cells in a given well that contained a heterogeneous population of B cells. This was achieved using the Fluorescent foci method. Briefly, Immunoglobulin-secreting B cells from a positive well were mixed with streptavidin beads (New England Biolabs) coated with biotinylated human FcRn and a 1:1200 final dilution of a goat anti-rat or mouse Fcy fragment-specific FITC conjugate (Jackson). After static incubation at 37° C. for 1 hour, antigen-specific B cells could be identified due to the presence of a fluorescent halo surrounding that B cell. These individual B cells, identified using an Olympus microscope, were then picked with an Eppendorf micromanipulator and deposited into a PCR tube. Fluorescent foci were generated from 268 selected wells. Antibody variable region genes were recovered from single cells by reverse transcription polymerase chain reaction (RT)-PCR using heavy and light chain variable region-specific primers. Two rounds of PCR were performed on an Aviso Onyx liquid handling robot, with the nested 2° PCR incorporating restriction sites at the 3′ and 5′ ends allowing cloning of the variable regions into a mouse γl IgG (VH) or mouse kappa (VL) mammalian expression vector. Paired heavy and light chain constructs were co-transfected into HEK-293 cells using Fectin 293 (Invitrogen) and cultured in 48-well plates in a volume of 1 ml. After 5-7 days expression, supernatants were harvested and antibody subjected to further screening.
PCR successfully recovered heavy and light chain cognate pairs from single B cells from 156 of the selected wells. DNA sequence analysis of the cloned variable region genes identified a number of unique families of recombinant antibody. Following expression, transient supernatants were interrogated in both human IgG FACS blocking (described above) and IgG recycling assays. In some cases, purified mouse γl IgG was produced and tested (data labeled accordingly).
The recycling assay used MDCK II cells over-expressing human FcRn and beta 2 microglobulin plated out at 25,000 cells per well of a 96 well plate. These were incubated overnight at 37° C., 5% CO₂. The cells were washed with HBSS+Ca/Mg pH 7.2+1% BSA and then incubated with 50μ1 of varying concentrations of HEK-293 transient supernatant or purified antibody for 1 hour at 37° C., 5% CO₂. The supernatant was removed and 500 ng/ml of biotinylated human IgG (Jackson) in 50 μl of HBSS+Ca/Mg pH 5.9 +1% BSA was added to the cells and incubated for 1 hour at 37° C., 5% CO₂. The cells were then washed three times in HBSS+Ca/Mg pH 5.9 and 100μ1 of HBSS+Ca/Mg pH 7.2 added to the cells and incubated at 37° C., 5% CO₂ for 2 hours. The supernatant was removed from the cells and analysed for total IgG using an MSD assay with an anti-human IgG capture antibody (Jackson) and a streptavidin-sulpho tag reveal antibody (MSD). The inhibition curve was analysed by non-linear regression to determine IC50 values. Based on performance in these assays a family of antibodies was selected comprising the six CDRs given in SEQ ID NOs 1 to 6. Antibody CA170_01638 had the best activity and was selected for humanization, as previously described in WO2015/071330.

### EXAMPLE 1

### Humanisation Method

Antibody CA170_01638 was humanised by grafting the CDRs from the rat antibody V-regions onto human germline antibody V-region frameworks. In order to recover the activity of the antibody, a number of framework residues from the rat V-regions were also retained in the humanised sequence. These residues were selected using the protocol outlined by Adair et al. (1991) (Humanised antibodies WO91/09967). Alignments of the rat antibody (donor) V-region sequences with the human germline (acceptor) V-region sequences are shown in FIGS. 9A and B, together with the designed humanised sequences. The CDRs grafted from the donor to the acceptor sequence are as defined by Kabat (Kabat et al., 1987), with the exception of CDR-H1 where the combined Chothia/Kabat definition is used (see Adair et al., 1991 Humanised antibodies. WO91/09967). Human V-region IGKV1-27 plus JK4 J-region (http://www.imgt.org/) was chosen as the acceptor for the light chain CDRs. Human V-region IGHV3-7 plus JH3 J-region (http://www.imgt.org/) was chosen as the acceptor for the heavy chain CDRs.

Genes encoding a number of variant heavy and light chain V-region sequences were designed and constructed by an automated synthesis approach by Entelechon GmbH. Further variants of both heavy and light chain V-regions were created by modifying the VH and VK genes by oligonucleotide-directed mutagenesis. These genes were cloned into a number of vectors to enable expression of humanised 1638 Fab or 1735h5.hFab-scFv.768 in E. coli and mammalian cells, respectively. The variant chains, and combinations thereof, were assessed for their potency relative to the parent antibody, their biophysical properties and suitability for downstream processing, leading to the selection of the gL7 light chain graft and gH33 heavy chain graft. The final selected gL7 and gH33 graft sequences are shown in FIGS. 9A and B, and SEQ ID NOs: 8 and 9 respectively. This V-region pairing was named 1638.g49.

The light chain framework residues in graft gL7 are all from the human germline gene, with the exception of residues 70 and 71 (Kabat numbering), where the donor residues Histidine (H70) and Tyrosine (Y71) were retained, respectively. Retention of these two residues was important for full potency of the humanised antibody or Fab. Residue 56 in CDRL2 of the gL7 graft was mutated from an Aspartic acid (D56) to a Glutamic acid (E56) residue, thus removing a potential Aspartic acid isomerization site from the gL7 sequence. The heavy chain framework residues in graft gH33 are all from the human germline gene, with the exception of residues 48 and 78 (Kabat numbering), where the donor residues Leucine (L48) and Alanine (A78) were retained, respectively. Retention of these two residues was essential for full potency of the humanised 1638.g49 Fab or 1735h5.hFab-scFv.768.

Another earlier graft, 1638.g28 was also generated and this contained more donor residues in the heavy chain (gH2) than the 1638.g49 graft (F24, L48, K71, T73, A78 and V93). Also the light chain of this antibody (gL2) contains the unmodified CDRL2 given in SEQ ID NO: 5 rather than the modified CDRL2 of SEQ ID NO: 7 which is used in 1638.g49. Sequences of both sets of antibodies are given in FIG. 8.

For expression of 1638.g49 Fab in E. coli, the humanised heavy and light chain V-region genes were cloned into the UCB expression vector pMXE811 , which contains DNA encoding the human C-kappa constant region (K1m3 allotype) , the human gamma-1 CH1 constant region with a truncated hinge (G1m17 allotype), and the E. coli chaperone proteins FkpA and DsbC.

A Fab-dsscFv fusion protein comprising the 1638.g49 variable domains was constructed and expressed essentially as described in Example 4 of WO2013/068571 using the heavy and light chain sequences given in FIG. 8, SEQ ID NO:12 and SEQ ID NO:10 respectively. For expression of the Fab-dsscFv fusion protein, termed 1735h5.hFab-scFv.768, in mammalian cells, the humanised light chain V-region gene was joined to a DNA sequence encoding the human C-kappa constant region (K1m3 allotype), to create a contiguous 1735h5.hFab-scFv.768 light chain gene. The humanised heavy chain V-region gene was joined to a DNA sequence encoding the human gamma-1 CH1 constant region domain. The heavy chain constant region was joined to a DNA sequence encoding a 4× GGGGS linker and an albumin binding dsscfv 645 gH5 gL4 to create a contiguous 1735h5.hFab-scFv.768 heavy chain gene. The heavy and light chain genes were cloned into a mammalian double gene expression vector pMXE755, to create 1735h5.hFab-scFv.768.

### EXAMPLE 2

### Preparation of 1735h5.hFab-scFv.768

1735h5.hFab-scFv.768 was expressed in a stable dihyrofolate reductase (DHFR) deficient Chinese Hamster Ovary cell line (CHO DG44). Cells were transfected using a Nuclefector (Lonza) following the manufactures instructions with a plasmid vector containing both the gene for DHFR as a selectable marker and the genes encoding the product. Transfected cells were selected in medium lacking hypoxanthine and thymidine, and in the presence of the DHFR inhibitor methotrexate. After culture of minipools up to shaker flask stage, growth and productivity were assessed and the highest expressing clones were chosen for evaluation in a fed-batch shake flask process. 8 L of culture was inoculated at a starting density of 0.3×10⁶ viable cells/mL and controlled at 36.8° C., in a 5% CO₂ atmosphere. Nutrient feeds were added from day 3 to 12 and glucose was added as a bolus addition when the concentration dropped below 5.8 g/L. The culture was harvested on day 14, via centrifugation at 4000×g for 60 min followed by 0.2 μm filtration.
Clarified cell culture supernatant from mini pool 4D4 was 0.22 μm sterile filtered and purified as follows. The filtered supernatant was loaded at <18 ml/min onto 450 ml GammabindPlus Sepharose XK50 Column (GE Healthcare) equilibrated in PBS pH7.4 (Sigma Aldrich Chemicals). After loading the column was washed with PBS pH7.4 and then eluted with 0.1M Glycine/HC1. pH2.7. The elution was followed by absorbance at 280 nm, the elution peak collected, and then neutralised with 2M Tris/HCl pH8.5. The neutralised samples were concentrated using a Vivaflow 50 Casette (Sartorious) with a 10 kDa molecular weight cut off membrane. An aliquot was analysed by size exclusion chromatography on a TSK gel G3000SWXL; 5 μm, 7.8×300 mm column developed with an isocratic gradient of 0.2M phosphate, pH7.0 at 1 ml/min, with detection by absorbance at 280nm. The % monomer was determined to be 90%. The bulk of the concentrated sample was applied to an XK50/60 Superdex200 column (GE Healthcare) equilibrated in PBS, pH7.4. The columns were developed with an isocratic gradient of PBS, pH7.4 at 10m1/min. Fractions were collected and analysed by size exclusion chromatography on a TSK gel G3000SWXL; 5 μm, 7.8×300 mm column developed with an isocratic gradient of 0.2M phosphate, pH7.0 at 1 ml/min, with detection by absorbance at 280 nm. Selected monomer fractions were pooled and concentrated to >20 mg/ml using Amicon Ultra-15 concentrators with a 10kDa molecular weight cut off membrane and centrifugation at 4000×g in a swing out rotor. Final samples were assayed; for concentration by A280 Scanning UV-visible spectrophotometer (Cary 50 Bio); for % monomer by size exclusion chromatography on a TSK gel G3000SWXL; 5 μm, 7.8×300 mm column developed with an isocratic gradient of 0.2M phosphate, pH7.0 at 1 ml/min, with detection by absorbance at 280nm; by reducing and non-reducing SDS-PAGE run on 4-20% Tris-Glycine 1.5 mm gels (Novex) at 50 mA (per gel) for 53 minutes; and for endotoxin by Charles River's EndoSafe® Portable Test System with Limulus Amebocyte Lysate (LAL) test cartridges.

### EXAMPLE 3

Additional Fab-dsscFv antibodies were designed using alternative anti-FcRn V-regions fixed in the Fab position, these were the 1519.g57 V regions described previously in WO2014/019727 and provided here in SEQ ID NOs:92 and 94 for the light and heavy chain domains respectively. This Fab was linked to an albumin binding dsscFv 645 gH5 gL4 (in the HL orientation (dsHL)), as described in Example 1. As shown in Table 3, these molecules were constructed as either a heavy chain (HC) Fab-dsscFv or a light chain (LC) Fab-dsscFv. For a HC Fab-dsscFv, the C-terminus of the CH1 region of the HC is linked to a dsscFv via a G₄S-based linker (11 amino acids) and, this HC is paired with a 1519 light cKappa chain (LC); similarly, for a LC Fab-dsscFv, the C-terminus of the cKappa region of the LC is linked to a dsscFv via a G₄S-based linker (11 amino acids), and this is paired with a 1519 CH1 HC (no hinge). A 1519 Fab no hinge (nh) was used as a control.

TABLE 3
Plasmids used in this study

| Antibody | Format | HC | LC |
|---|---|---|---|
| 1519 Fab | Fab nh | 1519 Fab HC nh | 1519 Fab LC |
| 1519 Fab-dsscFv 1 | HC Fab- | 1519 CH1-645 dsHL | 1519 Fab LC |
| | dsscFv | | |
| 1519 Fab-dsscFv 2 | LC Fab- | 1519 Fab | 1519 Fab LC- |
| | dsscFv | | 645 dsHL |

The genes were cloned into a proprietary mammalian expression vector under the control of a hCMV promoter and transfected into the CHO-S XE cell line (UCB) for transient expression using CD CHO media (Life Technologies) and 2 mM glutamax. The cultures were incubated in Kuhner shakers at 37° C., 8.0% CO₂, 140 rpm and when cells reached >2×10⁵ cells/ml (˜24 h), the T° C. was reduced to 32° C. On day 3 post-transfection, 3 mM sodium butyrate (Sigma-Aldrich) per L transfection was aseptically added to each flask. The cultures were incubated for a total of 14 days. The supernatant was harvested by centrifuging the culture at 4000 rpm for 1 h at 4° C. and filter-sterilized through a 0.2 μm filter. Expression titres were quantified by Protein G HPLC using a 1 ml GE HiTrap Protein G column (GE Healthcare) and Fab standards produced in-house. As shown in Table 4, a ˜1.5 fold increase in expression titres of HC Fab-scFv was observed when compared to LC Fab-scFv.

TABLE 4
Expression titres from transient expression in CHO S-XE cell line

| | Concentration. |
|---|---|
| Antibody | (mg/L) |
| 1519 Fab | 124 |
| Fab-dsscFv 1 | 188 |
| Fab-dsscFv 2 | 122 |

The antibody proteins were purified by Protein G affinity chromatography. Briefly, supernatants were loaded on a HiTrap Protein G (GE Healthcare) and then washed with PBS pH 7.4. The bound material was eluted with 0.1 M glycine pH 2.7, and neutralized with 2 M Tris-HCl (pH 8.5) prior to buffer exchange into PBS pH 7.4. The eluted protein was quantified by absorbance at 280 nm and stored at 4° C. for further analysis.
Size exclusion chromatography (SE HPLC) was used to determine the monomeric status of the antibody. Purified protein samples (˜20 μg) were loaded on to a TSKgel G3000SW, 10 μm, 7.5 mm ID×300 mm column (Tosoh) and developed with an isocratic gradient of 0.2 M phosphate pH 7 at 1 mL/min. Continuous detection was by absorbance at 280 nm. The monomer yield of each protein is given in Table 5.

TABLE 5
% monomer as determined by SE HPLC of antibodies

| Antibody | % Monomer |
|---|---|
| 1519 Fab | 55 |
| Fab-dsscFv 1 | 47 |
| Fab-dsscFv 2 | 40 |

For sodium dodecyl sulfate-polyacrylamide gel electrophoresis (SDS-PAGE) analysis, samples were prepared by adding 4×Novex NuPAGE LDS sample buffer (Life Technologies) and either 10× NuPAGE sample reducing agent (Life Technologies) or 100 mM N-ethylmaleimide (Sigma-Aldrich) to ˜2 μg purified protein of 1519 Fab or Fab-scFv, and were heated to 100° C. for 3 min. The samples were loaded onto a 15 well Novex 4-20% Tris-glycine SDS-polyacrylamide gel (Life Technologies) and separated at a constant voltage of 125 V for 110 min in Tris-glycine SDS running buffer (Life Technologies). Novex Mark12 wide-range protein standards (Life Technologies) were used as standards. The gel was stained with Coomassie Brilliant Blue (Sigma-Aldrich) in 10% methanol, 7.5% acetic acid for 1 h and destained with several changes of distilled water. 1519 Fab, with a theoretical molecular weight (MW) of ˜47 kDa was shown to have a faster mobility on non-reducing SDS-PAGE whereas the Fab-dsscFv on non-reduced gels were observed to migrate slower than their respective theoretical MWs (Table 6). This is not wholly unexpected, is an observation that has been previously reported in the literature and can be attributed to differential SDS binding and compact tertiary structures of proteins. Regardless when proteins were reduced, all proteins migrated at a mobility rate approaching their respective theoretical MWs (Table 6).

TABLE 6
MWs of proteins

| | Non-reduced | Non-reduced | Reduced | Reduced |
|---|---|---|---|---|
| | Theoretical | Observed | Theoretical | Observed |
| Antibody | MW (kDa) | MW (kDa) | MW (kDa) | MW (kDa) |
| 1519 Fab | ~47 | ~38 | ~24 (LC) | ~25 (LC) |
| | | | ~23 (HC) | ~23 (HC) |
| Fab-dsscFv 1 | ~75 | ~78 | ~24 (LC) | ~28 (LC) |
| | | | ~51 (HC) | ~50 (HC) |
| Fab-dsscFv 2 | ~75 | 76 | ~51 (LC) | ~50 (LC) |
| | | | ~23 (HC) | ~27 (HC) |

The theoretical MWs were calculated from the amino acid sequence using an algorithm provided in the Vector NTI software (Life Technologies). Observed MWs were calculated from a plot of the migration rates of the Mark 12 protein standards against MW.

### EXAMPLE 4

### Biophysical Properties of 1735h5.hFab-scFv.768

The 1735h5.hFab-scFv.768 molecule was subjected to a series of biochemical and biophysical analyses to screen for robustness of the molecule for development and administration stability. The analyses included mass spectrometry for confirmation of intact mass and disulphide arrangement, thermal stability (Tₘ) (melting temperature at mid-point of unfolding); experimental isoelectric point (pI) and charge variants, aggregation stability at an air-liquid interface (mimic of shear stress in manufacture) and high concentration and viscosity stability. The characteristics of 1735h5.hFab-scFv.768 were compared to equivalent Fab, FabFv and IgG4 molecules where possible, comprising the same variable region sequence as the Fab moiety of 1735h5.hFab-scFv.768.

### Mass Spectrometry Analysis.

(i) Both the intact and reduced masses of purified 1735h5.hFab-scFv.768 were obtained by mass spectrometry using an Agilent 6510 Q-Tof with a C8 enrichment column chip. The intact mass was obtained by diluting the sample to 0.15 mg/mL with 98% water (18.2 megohm), 2% ultra-grade methanol, 0.3% formic acid. The reduced masses were obtained by initially incubating 100 μl of the sample at 1 mg/mL with 10 mM TCEP at 37° C. for 45 minutes followed by dilution to 0.15 mg/mL with 98% water (18.2 megohm), 2% ultra-grade methanol, 0.3% formic acid The mobile phase A was 0.1% formic acid in water (18.2 megohm); mobile phase B was 80% iso-propanol, 20% acetonitrile, 0.1% formic acid. Data processing was carried using Agilent's MassHunter deconvolution software The intact and reduced masses were consistent with the theoretical values (Table 7)

TABLE 7
Intact and reduced mass analysis of 1735h5.hFab-
scFv.768 by mass spectrometry.

| | Theoretical Mass | Measured Mass |
|---|---|---|
| Species | (Da) | (Da) |
| Light Chain | 23503.32 | 23504.3 |
| Heavy Chain | 51179.9 | 51180.9 |
| Intact | 74679.19 | 74680.7 |

(ii) The disulphide pairing was confirmed by enzymatic digestion under non reducing conditions followed by mass spectrometric analysis. The digestion was performed by incubating 24 of purified 1735h5.hFab-scFv.768 (10 mg/mL) with 18 μL of 50 mM iodacetamide in 6M guanidine hydrochloride at 37° C. for 45 minutes, followed by the addition of 404 of 25 mM Tris(hydroxymethyl)aminomethane (Tris) pH 7.5 plus 1 μL of 0.5 mg/mL LysC (in suspension buffer provided). This mixture was further incubated at 37° C. for 70 minutes and then diluted with 1004 of 1 mM calcium chloride. Chymotrypsin (14 at 0.5 mg/mL in 1 mM hydrochloric acid) was added and the reaction mixture was incubated overnight at room temperature. The reaction was then quenched by the addition of 164 of 10% formic acid. The sample was then applied to a 1×150mm C18 reverse-phase column equilibrated with 95% solvent A : 5% solvent B (1:1 MeCN:1-propanol/0.1% formic acid) at 20 uL/min and 55° C. using an Acquity uPLC and Fusion Orbitrap mass spectrometer. Orbitrap MS data was collected in +ve-ion mode at 15000 resolution in the range 700-4000 m/z during elution. The data were processed using Thermo PepFinder software.
All of the predicted disulphide bonds for the 1735h5.hFab-scFv.768 molecule were observed, confirming correct intact disulphide pairing.
Thermal stability measurement (Tₘ)
The melting temperature was measured using Differential scanning calorimetry (DSC) using a Micro-Cal VP-Cap (GE Healthcare) in Dulbecco's phosphate-buffered saline buffer (PBS) pH 7.4 and compared to the Fab and Fab-Fv molecules previously described in WO2015/071330. The molecule was adjusted to 1 mg/ml using PBS pH 7.4 and the final concentration confirmed using absorbance at 280 nm using a Varian Cary 50-Bio spectrophotometer. The antibody molecules and buffer blanks were sampled from 96-well plates using the robotic attachment. Two scans with buffer blanks were performed for baseline subtraction. The analysis was performed from 20° C. to 110° C. at a scan rate of 1° C./minute using the passive feedback mode and were analyzed using Origin 7.0. (Microcal analysis software Version 2.0).
Two unfolding domains were observed (see FIG. 6) The first unfolding event at 78.9° C. corresponded to the scFv domain and the second unfolding event at 84.8° C. corresponded to the Fab domain. The melting temperature of the scFv was found to be 5.5° C. higher than the corresponding Fv, hence the scFv conferred greater stability to the 1735h5.hFab-scFv.768 format. The high melting temperature of the Fab domain conferring molecular stability of the molecule was comparable to that obtained for the parent Fab molecule and hence the format did not result in thermal instability of the Fab domain.
Experimental pI and analysis of charge variants
The experimental pI was measured using whole-capillary imaged cIEF ICE3 system (ProteinSimple). Samples were prepared by mixing the following: 30 μL sample (from a 1 mg/mL stock in HPLC grade water), 35 μL of 1% methylcellulose solution (Protein Simple), 4 μL pH3-10 ampholytes (Pharmalyte), 0.5 μL of 4.65 and 0.5 μL 9.77 synthetic pI markers (ProteinSimple), 12.5 μL of 8M urea solution (Sigma-Aldrich). HPLC grade water was used to make up the final volume to 100 μL. The mixture was vortexed briefly to ensure complete mixing and centrifuged at 10,000 rpm for 3 minutes to remove air bubbles before analysis. Samples were focused for 1 minute at 1.5 kV, followed by 5 minutes at 3 kV, and A₂₈₀ images of the capillary were taken using the ProteinSimple software. The resulting electropherograms were first analysed using ICE3 software and pI values were assigned (linear relationship between the pI markers). The calibrated electropherograms were then integrated using Empower software (Waters). The pI was found to be high, that is 9.11 and similar to the parent Fab molecule (9.22). The percentage of acidic/ basic species was low and comparable to the parent Fab molecule.

### Aggregation Propensity at an Air-Liquid Interface

A purified sample of 1735h5.hFab-scFv.768 (3×250 μL aliquots) in PBS pH 7.4 at 1 mg/mL was vortexed at 1400 rpm at 25° C. in 1.5 mL eppendorfs using an Eppendorf Mixmate. Samples were analysed for turbidity generation at various time-points post vortexing by obtaining absorption at 595 nm using a spectrophotometer (Varian). The mean of values 595 nm were plotted versus time. The aggregation propensity was found to be low up to 48 h vortexing and to be equivalent to the parent Fab. This shows that this format would exhibit similar aggregation stability through manufacturing steps comprising shear stress (ultra-filtration) as the equivalent Fab molecule. Both 1735h5.hFab-scFv.768 and the parent Fab demonstrated a slower aggregation rate compared to the equivalent IgG4 format.

### Effect of Concentration and Viscosity

To determine whether 1735h5.hFab-scFv.768 could be concentrated to >200 mg/mL, 4mL of purified material at 21.5 mg/mL in PBS pH 7.40 was concentrated to 218.6 mg/mL using an Amicon Ultra centrifugal filter unit Ultra-4 (molecular weight cut-off 10 kDa) at 3500 g for 45 minutes. Post filtration, the sample was analysed by Size Exclusion UPLC (2× Acquity BEH200 1.7 μm, 4.6 mm×150 mm columns in series, 0.3 mL/minute, isocratic, 0.2M Phosphate buffer pH 7.0) for detection of soluble aggregates or fragments; Dynamic light scattering (Malvern Nano ZS) for detection of insoluble particulate material and SDS PAGE (non reducing and reducing conditions using 4-20% Tris Glycine gels, 125 mV constant voltage) for aggregation and fragmentation changes. There was no evidence for aggregation or fragmentation as a consequence of concentration. Viscosity was measured at 218.6 mg/mL using a TA Instruments DHR-1 Discovery Hybrid Rheometer. The infinite rate viscosity was found to be 9.9±0.46 cP in the PBS pH 7.4 buffer (non-optimised pre-formulation buffer), this was lower than the equivalent Fab molecule in its chosen formulation buffer, that is 17.1±0.9 cP.It has been demonstrated that 1735h5.hFab-scFv.768 can be concentrated to a high concentration with low viscosity.
Overall, the 1735h5.hFab-scFv.768 molecule demonstrated good biophysical characteristics that were comparable to the parent Fab.

### EXAMPLE 5

### Affinity of 1735h5.hFab-scFv.768 for hFcRn Binding

Biomolecular interaction analysis using surface plasmon resonance technology (SPR) was performed on a Biacore T200 system (GE Healthcare) and binding to human FcRn extracellular domain determined. Human FcRn extracellular domain was provided as a non-covalent complex between the human FcRn alpha chain extracellular domain (SEQ ID NO: 21) and β2 microglobulin (β2M) (SEQ ID NO: 23). Affinipure F(ab′)₂ fragment goat anti-human IgG, F(ab′)₂-specific (Jackson ImmunoResearch Lab, Inc.) at 50 μg/ml in 10 mM NaAc, pH 5 buffer was immobilized on a CMS Sensor Chip via amine coupling chemistry to a capture level between 4000-5000 response units (RU) using HBS-EP⁺(GE Healthcare) as the running buffer.

50 mM Phosphate, pH6+150 mM NaCl+0.05% P20 or HBS-P⁺, pH7.4 (GE Healthcare) was used as the running buffer for the affinity assay. The antibody, 1735h5.hFab-scFv.768 was diluted to 0.25 μg/ml in running buffer and an injection 60 s at 10 μl/min was used for capture by the immobilized anti-human F(ab)₂. Human FcRn extracellular domain was titrated from 20 nM to 1.25 nM over the captured 1735h5.hFab-scFv.768 for 300 s at 30 μl/min followed by 600 s dissociation. The surface was regenerated at 10 μl/min by 2×60 s 50 mM HCl for the running buffer at pH6 or by 60 s 40 mM HCl and 30 s 10 mM NaOH for the running buffer at pH7.4. The data was analysed using Biacore T200 evaluation software (version 1.0) using the 1:1 binding model with local Rmax.

TABLE 8
Affinity data for anti-hFcRn 1735h5·hFab-scFv.768 at pH6.0 and pH7.4

| | pH6 | | | pH7.4 | | |
|---|---|---|---|---|---|---|
| Sample | ka (M⁻¹s⁻¹) | kd (s⁻¹) | KD (M) | ka (M⁻¹s⁻¹) | kd (s⁻¹) | KD (M) |
| 1 | 1.16E+06 | 1.84E−04 | 1.59E−10 | 8.93E+05 | 4.07E−05 | 4.56E−11 |
| 2 | 1.12E+06 | 1.79E−04 | 1.60E−10 | 8.49E+05 | 3.90E−05 | 4.59E−11 |
| 3 | 1.13E+06 | 1.83E−04 | 1.61E−10 | 8.56E+05 | 3.71E−05 | 4.34E−11 |
| 4 | 1.10E+06 | 1.87E−04 | 1.70E−10 | 8.57E+05 | 4.45E−05 | 5.19E−11 |
| 5 | 1.13E+06 | 1.71E−04 | 1.51E−10 | 8.53E+05 | 3.58E−05 | 4.20E−11 |
| Average | 1.13E+06 | 1.81E−04 | 1.60E−10 | 8.61E+05 | 3.94E−05 | 4.58E−11 |

The affinity of 1735h5.hFab-scFv.768 for human FcRn was therefore determined to be 160 pM at pH 6.0 and 46 pM at pH7.4.

### EXAMPLE 6

### Functional Cell Based Assays

FcRn expression is primarily intracellular (Borvak J et al. 1998, Int. Immunol., 10 (9) 1289-98 and Cauza K et al. 2005, J. Invest. Dermatol., 124 (1), 132-139), and associated with endosomal and lysosomal membranes. The Fc portion of IgG binds to FcRn at acidic pH (<6.5), but not at a neutral physiological pH (7.4) (Rhagavan M et al. 1995) and this pH-dependency facilitates the recycling of IgG.

Once it is taken up by pinocytosis and enters the acidic endosome, IgG bound to FcRn will be recycled along with the FcRn to the cell surface, whereas at the physiologically neutral pH the IgG will be released. (Ober RJ et al. 2004, The Journal of Immunology, 172, 2021-2029). Any IgG not bound to FcRn will enter the lysosomal degradative pathway.

An in vitro assay was established to examine the ability of 1735h5.hFab-scFv.768 to inhibit the IgG recycling capabilities of FcRn. Briefly, MDCK II clone 7 cells were incubated with biotinylated human IgG, in the presence and absence of 1735h5.hFab-scFv.768 in an acidic buffer (pH 5.9) to allow binding to FcRn. All excess antibody was removed and the cells incubated in a neutral pH buffer (pH 7.2) which allows release of surface-exposed, bound and internalised IgG into the supernatant. The inhibition of FcRn was followed using an MSD assay to detect the amount of IgG recycled and thus released into the supernatant.

FIG. 1 shows 1735h5.hFab-scFv.768 inhibits IgG recycling in MDCK II clone 7 cells. MDCK II clone 7 cells were plated at 25,000 cells per well in a 96 well plate and incubated overnight at 37° C., 5% CO₂. The following day, cells were washed once with HBSS⁺(Ca/Mg) pH 7.2+1% BSA. The cells were incubated with, in the presence and absence of 1735h5.hFab-scFv.768 in HBSS⁺(Ca/Mg) pH 5.9+1% BSA for 1 hour at 37° C., 5% CO₂ following an incubation with 1 μg/ml (500 ng/ml final concentration) of biotinylated human IgG (Jackson) for 1 hour at 37° C., 5% CO₂. The cells were washed with HBSS⁺pH 5.9 then incubated at 37° C., 5% CO₂ for 2 hours in HBSS⁺pH 7.2. The supernatant was removed from the cells and analysed for total IgG using an MSD assay (using an anti-human IgG capture antibody (Jackson) and a streptavidin-sulpho tag reveal antibody (MSD)). As shown in FIG. 1, which is a representative concentration response curve, 4 1735h5.hFab-scFv.768 inhibits IgG recycling in a concentration dependent manner with a mean EC₅₀ value (n=5) of 7.2 nM.

### EXAMPLE 7

### Cross Reactivity of 1735h5.hFab-scFv.768 with Non-human Primate FcRn

Biomolecular interaction analysis using surface plasmon resonance technology (SPR) was performed on a Biacore T200 system (GE Healthcare) and binding to Cynomolgus monkey FcRn extracellular domain determined. Cynomolgus monkey FcRn extracellular domain was provided as a non-covalent complex between the Cynomolgus monley FcRn alpha chain extracellular domain (SEQ ID NO: 17) and 132 microglobulin (β2M) (SEQ ID NO: 18). Affinipure F(ab′)₂ fragment goat anti-human IgG, F(ab′)₂-specific (Jackson ImmunoResearch Lab, Inc.) at 50 μg/ml in 10 mM NaAc, pH 5 buffer was immobilized on a CMS Sensor Chip via amine coupling chemistry to a capture level between 4000-5000 response units (RU) using HBS-EP⁺ (GE Healthcare) as the running buffer.

50 mM Phosphate, pH6+150 mM NaCl+0.05% P20 or HBS-P⁺, pH7.4 (GE Healthcare) was used as the running buffer for the affinity assay. The antibody, 1735h5.hFab-scFv.768 diluted to0.5 μg/ml in running buffer and an injection of 60 s at 10 μl/min was used for capture by the immobilized anti-human F(ab′)₂. Cynomolgus monkey FcRn extracellular domain was titrated from 20 nM to 1.25 nM over the captured 1735h5.hFab-scFv.768 for 300 s at 30 μl/min followed by 600 s dissociation. The surface was regenerated at 10 μl/min by 2×60 s 50 mM HCl for the running buffer at pH6 or by 60 s 40 mM HCl and 30 s 10 mM NaOH for the running buffer at pH7.4.

The data was analysed using Biacore T200 evaluation software (version 1.0) using the 1:1 binding model with local Rmax.

TABLE 9
Affinity data for anti-hFcRn 1735h5·hFab-scFv.768 for Cynomolgus
monkey FcRn at pH6.0 and pH7.4

| | pH6 | | | pH7.4 | | |
|---|---|---|---|---|---|---|
| Sample | ka (M⁻¹s⁻¹) | kd (s⁻¹) | KD (M) | ka (M⁻¹s⁻¹) | kd (s⁻¹) | KD (M) |
| 1 | 1.26E+06 | 1.82E−04 | 1.44E−10 | 1.01E+06 | 5.88E−05 | 5.85E−11 |
| 2 | 1.26E+06 | 1.97E−04 | 1.56E−10 | 9.99E+05 | 4.33E−05 | 4.34E−11 |
| 3 | 1.26E+06 | 1.98E−04 | 1.58E−10 | 1.00E+06 | 4.64E−05 | 4.64E−11 |
| 4 | 1.24E+06 | 2.10E−04 | 1.70E−10 | 1.01E+06 | 4.16E−05 | 4.12E−11 |
| 5 | 1.24E+06 | 2.00E−04 | 1.61E−10 | 1.01E+06 | 3.60E−05 | 3.57E−11 |
| Average | 1.25E+06 | 1.97E−04 | 1.58E−10 | 1.00E+06 | 4.52E−05 | 4.50E−11 |

The affinity of 1735h5.hFab-scFv.768 for Cynomolgus monkey FcRn was therefore determined to be 158 pM at pH 6.0 and 45 pM at pH7.4.

### EXAMPLE 8

### 1735h5.hFab-scFv.768 Treatment Enhances the Clearance of hIgG In Vivo in hFcRn Transgenic Mice

The effect of 1735h5.hFab-scFv.768 on the clearance of human IVIG was determined in human FcRn transgenic mice (B6.Cg-Fcgrt^{tm1Der} Tg(FCGRT)32Dcr/DcrJ, JAX Mice). Mice were infused intravenously with 500 mg/kg human IgG (Human IgI 10% Gamunex-c, Talecris Biotherapeutics). 24 hours later animals were dosed with vehicle control (PBS) or anti-FcRn intravenously as a single dose (100 mg/kg). Serial tail tip blood samples were taken at −1, 8, 24, 48, 72, 96, 144 and 192 hours relative to anti-FcRn treatment. Serum levels of human IgG in hFcRn mice were determined by LC-MS/MS. Data presented in FIG. 2 are mean ±SEM with 5-6 mice per 1735h5.hFab-scFv.768 treatment group and 2 mice for PBS vehicle control. Blocking of hFcRn by 1735h5.hFab-scFv.768 resulted in accelerated clearance of hIVIG and lower concentrations of total IgG were observed compared to control mice. These reduced IgG concentrations were significantly different (p<0.01), for all doses of 1735h5.hFab-scFv.768, vs control mice from 24 hrs until the end of the experiment. Significance was measured by one way ANOVA and Tukeys post test.

Although mouse IgG did not bind to the human FcRn present in these transgenic mice, endogenous mouse albumin did bind and was recycled by the human FcRn. Although binding of anti-human FcRn to human FcRn does not block binding of albumin to FcRn in an vitro assay, given the albumin-binding properties of 1735h5.hFab-scFv.768, the effect on clearance of endogenous mouse albumin was evaluated. Data are shown in FIG. 3. Since albumin concentration in serum was somewhat variable (from 16.6 to 59.9 mg/mL in a group of 30 mice, prior to injection of anti-FcRn drug), to allow easier comparison of group results, albumin data were normalised and given as a percentage of the serum albumin concentration at time zero in FIG. 3. 1735h5.hFab-scFv.768 had a modest effect on albumin concentration which was maximal at about 25% at the 100 and 30 mg/kg doses after 72 hours and showed no effect at 10 mg/kg. The effect appeared to be reversible.

### EXAMPLE 9

### 1735h5.hFab-scFv.768 Treatment Shows Pharmacokinetics Similar to that of an IgG and Superior to the Expected PK of a Fab Fragment in Mice

The PK of 1735h5.hFab-scFv.768 was determined in normal mice. A 10 mg/kg IV bolus dose of 1735h5.hFab-scFv.768 was administered to twelve male C57/B16 mice. Plasma samples were taken at selected intervals from four animals per time point. Plasma concentrations of 1735h5.hFab-scFv.768 were determined by an LC/MS-MS assay for a proteotypic peptide of 1735h5.hFab-scFv.768. Plasma pharmacokinetic parameters were derived using noncompartmental analysis. The data in FIG. 4 show that 1735h5.hFab-scFv.768 has PK properties comparable to those of a typical human IgG dosed in mice. The distribution volume is similar to plasma volume (34 mL/kg), the terminal halflife (t_{1/2})is 4.1 days and the plasma clearance (CL)is 14 mL/day/kg.

The PK of 1735h5.hFab-scFv.768 was also evaluated in human FcRn transgenic mice in the study described in Example 7 (B6.Cg-Fcgrt^{tm1Der} Tg(FCGRT)32Dcr/DcrJ, JAX Mice infused intravenously with 500 mg/kg human IgG and subsequently dosed with 1735h5.hFab-scFv.768. Serial tail tip blood samples were taken at −24, 8, 24, 48, 72, 96, 144 and 192 hours relative to 1735h5.hFab-scFv.768 treatment and serum levels of 1735h5.hFab-scFv.768 were determined by LC-MS/MS. Data presented in FIG. 5 are mean ±SEM from 5 mice per treatment group and showed that the PK properties of 1735h5.hFab-scFv.768 were as least as good as a whole IgG anti-FcRn molecule with comparable potency and affinity.

1. An anti-FcRn antibody fusion protein comprising a Fab fragment linked directly or via a linker to a scFv wherein the Fab fragment binds to FcRn and the scFv binds to a serum carrier protein.

2. An anti-FcRn antibody fusion protein comprising a Fab fragment linked directly or via a linker to a scFv wherein the Fab fragment comprises a heavy chain and a light chain wherein the variable region of the heavy chain comprises three CDRs, wherein CDR H1 has the sequence given in SEQ ID NO: 1, CDR H2 has the sequence given in SEQ ID NO: 2, and CDR H3 has the sequence given in SEQ ID NO: 3 and the variable region of the light chain comprises three CDRs, wherein CDR L1 has the sequence given in SEQ ID NO: 4, CDR L2 has the sequence given in SEQ ID NO: 5 or SEQ ID NO: 7 and CDR L3 has the sequence given in SEQ ID NO: 6.

3. An anti-FcRn antibody fusion protein according to claim 2 wherein the heavy chain of the Fab fragment comprises the sequence given in SEQ ID NO:9 and the light chain of the Fab fragment comprises the sequence given in SEQ ID NO: 8.

4. An anti-FcRn antibody fusion protein according to claim 2 wherein the heavy chain of the Fab fragment comprises the sequence given in SEQ ID NO:14 and the light chain of the Fab fragment comprises the sequence given in SEQ ID NO: 10.

5. An anti-FcRn antibody fusion protein according to claim 1 or 2 wherein the scFv binds albumin.

6. An anti-FcRn antibody fusion protein according to claim 5 wherein the scFv binds human serum albumin.

7. An anti-FcRn antibody fusion protein according to claim 1 or 2 wherein the heavy and light chain variable domains of the scFv are linked by any suitable linker, such as that given in SEQ ID NO:30.

8. An anti-FcRn antibody fusion protein according to claim 1 or 2 wherein the scFv is linked directly or via a linker to the C-terminus of the heavy or the light chain of the Fab fragment.

9. An anti-FcRn antibody fusion protein according to claim 1 or 2 wherein the scFv is linked to the C-terminus of the heavy chain of the Fab fragment via a linker having the sequence given in SEQ ID NO:33.

10. An anti-FcRn antibody fusion protein according to claim 1 or 2 wherein the scFv comprises a heavy chain variable domain comprising three CDRs, wherein CDR H1 has the sequence given in SEQ ID NO: 85, CDR H2 has the sequence given in SEQ ID NO: 86, and CDR H3 has the sequence given in SEQ ID NO: 87 and a light chain variable domain comprising three CDRs, wherein CDR L1 has the sequence given in SEQ ID NO: 88, CDR L2 has the sequence given in SEQ ID NO: 89 and CDR L3 has the sequence given in SEQ ID NO: 90.

11. An anti-FcRn antibody fusion protein according to claim 1 or 2 in which the scFv is a disulphide stabilised scFv (dsscFv).

12. An anti-FcRn antibody fusion protein according to claim 11 wherein the dsscFv comprises a heavy chain variable domain comprising the sequence given in SEQ ID NO: 15 and a light chain variable domain comprising the sequence given in SEQ ID NO: 16.

13. An anti-FcRn antibody fusion protein according to claim 12 having a heavy chain comprising the sequence given in SEQ ID NO:12 and a light chain comprising the sequence given in SEQ ID NO: 10.

14. An anti-FcRn antibody fusion protein which binds human FcRn comprising a heavy chain, wherein the heavy chain comprises a sequence having at least 80% identity or similarity to the sequence given in SEQ ID NO: 12 and wherein the light chain comprises a sequence having at least 80% identity or similarity to the sequence given in SEQ ID NO: 10.

15. An anti-FcRn fusion protein according to claim 14 wherein the antibody Fab fragment has the sequence given in SEQ ID NO: 1 for CDR-H1, the sequence given in SEQ ID NO: 2 for CDR-H2, the sequence given in SEQ ID NO: 3 for CDR-H3, the sequence given in SEQ ID NO: 4 for CDR-L1, the sequence given in SEQ ID NO: 5 or SEQ ID NO: 7 for CDR-L2 and the sequence given in SEQ ID NO: 6 for CDR-L3.

16. An isolated DNA sequence encoding the heavy and/or light chain(s) of an antibody fusion protein according to claim 2 or 14.

17. A cloning or expression vector comprising one or more DNA sequences according to claim 16.

18. A vector according to claim 17 wherein the vector comprises the sequence given in SEQ ID NO: 11 and the sequence given in SEQ ID NO: 13.

19. A host cell comprising one or more cloning or expression vectors according to claim 17.

20. A process for the production of an antibody fusion protein having binding specificity for human FcRn, comprising culturing the host cell of claim 19 and isolating the antibody fusion protein.

21. A pharmaceutical composition comprising an anti-FcRn antibody fusion protein as defined in claim 1, 2 or 14 in combination with one or more of a pharmaceutically acceptable excipient, diluent or carrier.

22. A pharmaceutical composition according to claim 21 comprising other active ingredients.

23. (canceled)

24. (canceled)

25. A method of treating a patient having an autoimmune disease comprising administering a therapeutically effective amount of an antibody or binding fragment thereof as defined in claim 1, 2, or 14.

26. A method according to claim 25 wherein the autoimmune disease is myasthenia gravis, Pemphigus vulgaris, Neuromyelitis optica, Guillain-Barré syndrome, lupus, idiopathic thrombocytopenic purpura or thrombotic thrombocytopenic purpura.

27. A method of treating a patient having an autoimmune disease comprising administering a therapeutically effective amount of a composition as defined in claim 21.

28. A method according to claim 27 wherein the autoimmune disease is myasthenia gravis, Pemphigus vulgaris, Neuromyelitis optica, Guillain-Barré syndrome, lupus, idiopathic thrombocytopenic purpura or thrombotic thrombocytopenic purpura.
